SEQUENCE LISTING (26 sequences)

SEQ ID NO: 1 - DNA, 1434 bp, Homo sapiens
atggactggacctggaggttcctctttgtggtggcagcagctacaggtgtccagtcccaggtccagctggtgcagtctggggctgaggtgaagaagcctgggtcctcggtgaaggtctcctgcaaggcttctggagacaccttcagcagccatgctatcacctgggtgcgacaggcccctggacaagggcttgagtggatgggcaggatcatccccatctttagtatttcagactacgcacagaagttccagggcagagtcacgtttactgcggacaaatccacgagcatagccttcatggagctgagcagcctgagatctcaggacacggccgtctattactgtgcgagatcattgctggcatattgtactggtggtagttgctccgaattcggggcggaagaccactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 477 aa, Homo sapiens
MDWTWRFLFVVAAATGVQSQVQLVQSGAEVKKPGSSVKVSCKASGDTFSSHAITWVRQAPGQGLEWMGRIIPIFSISDYAQKFQGRVTFTADKSTSIAFMELSSLRSQDTAVYYCARSLLAYCTGGSCSEFGAEDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 128 aa, Homo sapiens
QVQLVQSGAEVKKPGSSVKVSCKASGDTFSSHAITWVRQAPGQGLEWMGRIIPIFSISDYAQKFQGRVTFTADKSTSIAFMELSSLRSQDTAVYYCARSLLAYCTGGSCSEFGAEDHWGQGTLVTVSS

SEQ ID NO: 4 - protein, 5 aa, Homo sapiens
SHAIT

SEQ ID NO: 5 - protein, 17 aa, Homo sapiens
RIIPIFSISDYAQKFQG

SEQ ID NO: 6 - protein, 19 aa, Homo sapiens
SLLAYCTGGSCSEFGAEDH

SEQ ID NO: 7 - DNA, 711 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctttggtgcatccagcagggccactggcatcccagacaggttcagtggcagtgggtctgggacagacttcactctcaccatcagcagactggagcctgaagattttgcagtgtattactgtcagcagtatggtagctcaattcccgtgacgttcggccaagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 236 aa, Homo sapiens
METPAQLLFLLLLWLPDTTGEIVLTQSPGTLSLSPGERATLSCRASQSVSSNYLAWYQQKPGQAPRLLIFGASSRATGIPDRFSGSGSGTDFTLTISRLEPEDFAVYYCQQYGSSIPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 109 aa, Homo sapiens
EIVLTQSPGTLSLSPGERATLSCRASQSVSSNYLAWYQQKPGQAPRLLIFGASSRATGIPDRFSGSGSGTDFTLTISRLEPEDFAVYYCQQYGSSIPVTFGQGTKVEIK

SEQ ID NO: 10 - protein, 12 aa, Homo sapiens
RASQSVSSNYLA

SEQ ID NO: 11 - protein, 7 aa, Homo sapiens
GASSRAT

SEQ ID NO: 12 - protein, 10 aa, Homo sapiens
QQYGSSIPVT

SEQ ID NO: 13 - DNA, 1410 bp, Homo sapiens
atggaattggggctgagctgggttttccttgttgctcttttagaaggtgtccactgtgaggtgcaactggtggagtctgggggaggcttggtccagccgggggggtccctcagactctcctgtgcagcctctggactcaccattaatagctattggatgaactgggtccgccaaattccagggaaggggctggagtgggtggccaacataaacccaactggaagtcagcaatattatgtggactctgtgaggggccgattcaccatctccagagacgacgccaagaagtcactgtttctgcaattggacagcctcagagtcgaggacacggctgtgtattactgtgcgagaggccgtcggggtaactccgcctactgtgattcctggggccagggaacccaggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 469 aa, Homo sapiens [sequence omitted]

SEQ ID NO: 15 - protein, 120 aa, Homo sapiens
EVQLVESGGGLVQPGGSLRLSCAASGLTINSYWMNWVRQIPGKGLEWVANINPTGSQQYYVDSVRGRFTISRDDAKKSLFLQLDSLRVEDTAVYYCARGRRGNSAYCDSWGQGTQVTVSS

SEQ ID NO: 16 - protein, 5 aa, Homo sapiens
SYWMN

SEQ ID NO: 17 - protein, 17 aa, Homo sapiens
NINPTGSQQYYVDSVRG

SEQ ID NO: 18 - protein, 11 aa, Homo sapiens
GRRGNSAYCDS

SEQ ID NO: 19 - DNA, 708 bp, Homo sapiens
atggcctgggctctgctcctcctcagcctcctcactcagggcacaggatcctgggctcagtctgccctgactcagcctcgctcagtgtccggttcccctggacagtcagtcaccatctcctgcactggaaccagtagtgatattggtgcatatgattatgtctcgtggtatcgacaccaccccggcaaagcccccaaactcatcatttttgatgtcgccaagtggccctcaggggtcccagatcgcttctctggctccaagtctggcaacacggcctccctgaccatctctgggctccaggatgacgatgagggcgattattactgctgctcatatgcgggaagattcagtctaatattcggcggggggaccaagttgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 20 - protein, 235 aa, Homo sapiens
MAWALLLLSLLTQGTGSWAQSALTQPRSVSGSPGQSVTISCTGTSSDIGAYDYVSWYRHHPGKAPKLIIFDVAKWPSGVPDRFSGSKSGNTASLTISGLQDDDEGDYYCCSYAGRFSLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 111 aa, Homo sapiens
QSALTQPRSVSGSPGQSVTISCTGTSSDIGAYDYVSWYRHHPGKAPKLIIFDVAKWPSGVPDRFSGSKSGNTASLTISGLQDDDEGDYYCCSYAGRFSLIFGGGTKLTVLG

SEQ ID NO: 22 - protein, 14 aa, Homo sapiens
TGTSSDIGAYDYVS

SEQ ID NO: 23 - protein, 7 aa, Homo sapiens
DVAKWPS

SEQ ID NO: 24 - protein, 10 aa, Homo sapiens
CSYAGRFSLI

SEQ ID NO: 25 - protein, 2710 aa, Homo sapiens
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGLQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 26 - protein, 2366 aa, Homo sapiens
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

(19) United States
(12) Patent Application Publication
(10) Pub. No.: US 2015/0259402 A1
(43) Pub. Date: Sep. 17, 2015

(54) HUMAN ANTIBODY SPECIFIC TO TOXIN PRODUCED FROM CLOSTRIDIUM DIFFICILE, OR ANTIGEN-BINDING FRAGMENT THEREOF

(71) Applicant: EVEC INC., Sapporo-shi, Hokkaido (JP)

(72) Inventors: Kenzo Takada, Hokkaido (JP); Masahiro Watanabe, Hokkaido (JP); Takashi Torashima, Hokkaido (JP)

(21) Appl. No.: 14/436,057

(22) PCT Filed: Oct. 18, 2013
(86) PCT No.: PCT/JP2013/078326

§ 371 Date: Apr. 15, 2015

(30) Foreign Application Priority Data
Oct. 19, 2012 (JP) 2012-232323

(51) Int. Cl. C07K 16/12; A61K 39/08

(52) U.S. Cl. CPC C07K 16/1282; A61K 39/08; C07K 2317/76; C07K 2317/565; C07K 2317/92; A61K 2039/507

(57) ABSTRACT

The present invention provides novel human-derived monoclonal antibodies specifically binding to toxins produced by Clostridium difficile (toxin A and toxin B), respectively, and having excellent neutralizing activity, and antigen-binding fragments thereof. The present invention also provides a pharmaceutical composition for the treatment of Clostridium difficile infection comprising any of the antibodies or antigen-binding fragments thereof.

### TECHNICAL FIELD

The present invention relates to human-derived monoclonal antibodies specifically binding to toxins produced by Clostridium difficile (toxin A and toxin B), respectively, and antigen-binding fragments thereof, and a pharmaceutical composition for the treatment of Clostridium difficile infection comprising any of the antibodies or antigen-binding fragments thereof.

### BACKGROUND ART

In recent years, Clostridium difficile infection (hereinafter, abbreviated to CDI) derived from Clostridium difficile (hereinafter, referred to as C. difficile) has occurred frequently in hospitals, senior-citizen housing facilities, etc., in Europe and the United States. C. difficile has received attention as a pathogen responsible for hospital-acquired infection, as with MRSA (methicillin-resistant Staphylococcus aureus) and the like. The 2012 report (http://www.cdc.gov/vitalsigns/hai/) of the U.S. Centers for Disease Control and Prevention (CDC) estimates a yearly medical cost of a billion dollars for CDI in the United States and states that the number of yearly deaths in CDI reaches 14,000 people. In 2008, a medical cost (estimate) per patient was as very high as 2,871 to 4,846 dollars for first-time cases and 13,655 to 18,067 dollars for recurrent cases (Non Patent Literature 1).

C. difficile, a bacterium belonging to the genus Clostridium, is an obligate anaerobe that cannot grow in the presence of oxygen at a concentration of atmospheric level and forms spores. Other bacteria belonging to the genus Clostridium include highly pathogenic bacterial species such as Clostridium tetani (tetanus bacillus) and Clostridium botulinum. In the bacterial name Clostridium difficile, the term “difficile” is derived from difficulty to separate and culture because C. difficile is an obligate anaerobe. C. difficile has properties such as unresponsiveness to alcohol disinfection and unresponsiveness to many broad-spectrum antibiotics, and further, a cause of pseudomembranous colitis induced by antibiotics, and these features are considered to be partly responsible for the spread of hospital-acquired C. difficile infection (Non Patent Literatures 2, and 3).

CDI is found in every age group, but has a high incidence, particularly, in senior citizens and immune-compromised people. This is probably because broad-spectrum antibiotics are highly frequently used for most of these patients in order to treat various infections; thus, C. difficile resistant to these antibiotics grows as a result of microbial substitution in the intestine to produce toxins such as toxin A and toxin B, inducing symptoms such as diarrhea. It has been reported that: patients infected by C. difficile have a low antibody titer of an anti-toxin A antibody in serum and develop diarrhea with high frequency (Non Patent Literature 4): and, as for recurrence, the frequency of recurrence is high when the antibody titer of an anti-toxin A antibody in serum is low (Non Patent Literature 5), suggesting that antibodies against C. difficile toxins play an important role in protecting against CDI.

The toxins produced by C. difficile are mainly toxin A and toxin B. Genes of these toxins are called tcdA and tcdB, respectively, and their gene products are called toxin A (or TcdA) and toxin B (TcdB), respectively. C. difficile strains are also classified on the basis of the productivity of these toxins. For example, a toxin A-positive/toxin B-positive strain, a toxin A-negative/toxin B-positive strain, and a toxin A-negative/toxin B-negative strain have been reported. Among them, the toxin A-positive/toxin B-positive strain and the toxin A-negative/toxin B-positive strain are known to cause diarrhea or enteritis (Non Patent Literatures 6 and 7). Recently, an increased number of a highly toxic bacterial strain called Clostridium difficile BUNAP1/027 (PCR ribotype 027/ST1; hereinafter, referred to as type 027) has been detected in many medical facilities in the United States, Canada, Europe, etc., and is also responsible for the outbreak of CDI. Since the type 027 bacterial strain produces a larger amount of toxin A or toxin B, patients infected by this strain are found more severe. In Japan as well, it was reported in 2005 that type 027 C. difficile was separated from the feces of a 30-year-old female patient with pseudomembranous colitis. A. S. Walker et al. in investigation conducted on CDI patients in the U.K. from September 2006 to May 2011 have further revealed the presence of PCR ribotype 078/ST11 (hereinafter, referred to as type 078), which is more lethal than type 027. In addition, this report indicates that patients infected by type 078 appear with a frequency as high as approximately 1/10 of people infected by type 027 (Non Patent Literature 8), suggesting that the need to develop a therapy of CDI has been increasingly urgent.

Most of CDI cases occur highly frequently during the long-term use of antibiotics, and 20 to 30% of diarrhea associated with the administration of antibiotics is reportedly attributed to C. difficile. CDI may cause pseudomembranous colitis, which forms a pseudomembrane in the digestive tract, and may remain merely at a mild diarrheal episode or may become severe to cause intestinal obstruction, perforation of the digestive tract, or sepsis, leading to death. Some CDI patients may experience repeated recurrence. Such recurrence may be attributed to the same bacterial strain as the initial C. difficile strain or may be attributed to a strain different therefrom. In HIV-infected individuals, C. difficile also serves as a primary pathogen that causes bacterial diarrhea.

Therapeutic drugs effective for CDI are limited to metronidazole and vancomycin. Vancomycin, however, might induce the vancomycin resistance of C. difficile in the intestine, while metronidazole has neurotoxicity. In consideration of these, it is preferred to avoid using repetitively or for a long period metronidazole or vancomycin at the time of recurrence.

Against this backdrop, many studies are underway toward the early establishment of a further therapy effective for CDI. In this respect, there have been reports on, for example, Ramoplanin (Non Patent Literature 9) and fidaxomicin (Non Patent Literature 10) as antibiotics effective for C. difficile infection, and a report on, for example, the maintenance or reconstruction of bacterial flora in the intestine by a yeast Saccharomyces boulardii (Non Patent Literature 2) as probiotics (which is a term introduced in contrast to antibiotics and refers to microbes having favorable influence on human bodies, as with, for example, certain kinds of lactic acid bacteria, or products or food products comprising the microbes), and further a report on a toxoid vaccine of toxin A and toxin B deactivated by formalin (Patent Literature 1). Also, there have been reports on a polyclonal antibody against toxin A and toxin B (Patent Literature 2), and monoclonal antibodies against these toxins (Patent Literatures 3, 4, and 5 and Non Patent Literatures 11 and 12). Particularly, monoclonal antibodies 3D8 (also called CDA1) (Patent Literature 3 and Non Patent Literature 12) and MDX1388 (also called 124-152 or CDB1) (Patent Literature 3 and Non Patent Literature 12) against toxin A and toxin B, respectively, joint-developed by the University of Massachusetts and Medarex, Inc. are currently under clinical trial (MK-3415A) for their combined use by Merck KGaA (Non Patent Literature 11).

### CITATION LIST

### Patent Literature

Patent Literature 1: WO2005/058353
Patent Literature 2: WO2010/094970
Patent Literature 3: WO2006/121422
Patent Literature 4: WO2006/071877
Patent Literature 5: WO2011/130650

### NON PATENT LITERATURE

Non Patent Literature 1: J. Hospital. Infect., 2010, (vol. 74) p. 309
Non Patent Literature 2: Guide to the Elimination of Clostridium difficile in Healthcare Settings (2008)
Non Patent Literature 3: J. Med. Microbiol., 2005, (vol. 54), p. 101
Non Patent Literature 4: N. Engl. J. Med., 2000, (vol. 342) p. 390
Non Patent Literature 5: Lancet. 2001 (357) p. 189
Non Patent Literature 6: J. Med. Microbiol., 2005, (vol. 54), p. 113
Non Patent Literature 7: Clin. Microbial. Rev., 2005, (vol. 18) p. 247
Non Patent Literature 8: Clin. Infect. Dis., 2013, (vol. 56) p. 1589
Non Patent Literature 9: J. Antimicro. Chemo., 2003, (vol. 51), Suppl. S3, iii31.iii35
Non Patent Literature 10: N. Engl. J. Med., 2011 (vol. 364) p. 422
Non Patent Literature 11: N. Engl. J. Med., 2010 (vol. 362) p. 197
Non Patent Literature 12: Inf. Immunity 2006, (vol. 74) p. 6339

### SUMMARY OF INVENTION

### Technical Problem

CDI is known to bring about a serious gastrointestinal disease in many cases as a result of growth of the bacterium in the digestive tract due to microbial substitution in the intestine caused by antibiotic treatment. In recent years, the spread of hospital-acquired infection by a strongly toxic bacterial strain such as Clostridium difficile BI/NAP1/027 and a large number of deaths in this infection have been reported, particularly, in Europe and the United States and have become urgent problems. Under these circumstances, monoclonal antibodies (3D8 and MDX1388) against toxin A and toxin B, respectively, are under clinical research on their combination therapy. Both of these antibodies are still susceptible to improvement in light of their affinity, neutralizing activity, and clinical trials results. For information, results of phase II clinical trial conducted on approximately 200 patients showed significant effects on the rate of recurrence of CDI in an antibody administration group compared with a placebo group, but no significant difference between these groups in terms of the incidence and duration of serious diarrheal episodes, the length of hospitalization of patients, a death rate, etc. (Non Patent Literature 11).

A great majority of antibody drugs approved as medicines are chimeric or humanized antibodies having problems associated with immunogenicity. In nature, completely human-derived antibodies are desirable as antibody drugs. In addition, even the completely human antibody drugs are required to provide pharmaceutical compositions having high neutralizing ability and remarkable therapeutic effects.

### Solution to Problem

Under these circumstances, the present inventors have conducted diligent studies with the aim of preparing human-derived monoclonal antibodies that exhibit high effectiveness. As a result, the present inventors have successfully prepared an anti-toxin A antibody that recognizes an epitope different from that for 3D8 and hPA-50 (Patent Literature 5) and is characterized in very high affinity and neutralizing activity, and have also successfully prepared an anti-toxin B antibody that recognizes an epitope different from that for MDX1388, and hPA-41 (Patent Literature 5) and has high neutralizing activity. The present inventors have further confirmed in vivo that the combined administration of these human antibodies remarkably protects in a dose-dependent manner against lethal action associated with C. difficile infection. In this way, the present invention has been completed.

Specifically, the present invention relates to, as described below, a monoclonal antibody specific for toxin A or an antigen-binding fragment thereof, a nucleic acid (polynucleotide) encoding the antibody or the antigen-binding fragment thereof, a vector containing the nucleic acid, and a host cell containing the vector. The present invention also relates to a monoclonal antibody specific for toxin B or a binding fragment thereof, a nucleic acid (polynucleotide) encoding the antibody or the antigen-binding fragment thereof, a vector containing the nucleic acid, and a host cell containing the vector. The present invention further relates to a pharmaceutical composition comprising the antibody of the present invention or the antigen-binding fragment.

[1] An antibody capable of specifically binding to Clostridium difficile toxin A protein and neutralizing its biological activity, or an antigen-binding fragment thereof, the antibody containing:
(i)
(a) heavy chain CDR1 comprising the amino acid sequence of SEQ ID NO: 4 or an amino acid sequence deviated from the amino acid sequence of SEQ ID NO: 4 by deletion, substitution, insertion, and/or addition mutations of one to several amino acid residues,
(b) heavy chain CDR2 comprising the amino acid sequence of SEQ ID NO: 5 or an amino acid sequence deviated from the amino acid sequence of SEQ ID NO: 5 by deletion, substitution, insertion, and/or addition mutations of one to several amino acid residues, and
(c) heavy chain CDR3 comprising the amino acid sequence of SEQ ID NO: 6 or an amino acid sequence deviated from the amino acid sequence of SEQ ID NO: 6 by deletion, substitution, insertion, and/or addition mutations of one to several amino acid residues; and
(ii)
(a) light chain CDR1 comprising the amino acid sequence of SEQ ID NO: 10 or an amino acid sequence deviated from the amino acid sequence of SEQ ID NO: 10 by deletion, substitution, insertion, and/or addition mutations of one to several amino acid residues,
(b) light chain CDR2 comprising the amino acid sequence of SEQ ID NO: 11 or an amino acid sequence deviated from the amino acid sequence of SEQ ID NO: 11 by deletion, substitution, insertion, and/or addition mutations of one to several amino acid residues, and
(c) light chain CDR3 comprising the amino acid sequence of SEQ ID NO: 12 or an amino acid sequence deviated from the amino acid sequence of SEQ ID NO: 12 by deletion, substitution, insertion, and/or addition mutations of one to several amino acid residues.
[2] The antibody or an antigen-binding fragment thereof according to [1], comprising:
(i)
(a) the amino acid sequence of heavy chain CDR1 comprising the amino acid sequence of SEQ ID NO: 4,
(b) the amino acid sequence of heavy chain CDR2 comprising the amino acid sequence of SEQ ID NO: 5, and
(c) the amino acid sequence of heavy chain CDR3 comprising the amino acid sequence of SEQ ID NO: 6; and
(ii)
(a) the amino acid sequence of light chain CDR1 comprising the amino acid sequence of SEQ ID NO: 10,
(b) the amino acid sequence of light chain CDR2 comprising the amino acid sequence of SEQ ID NO: 11, and
(c) the amino acid sequence of light chain CDR3 comprising the amino acid sequence of SEQ ID NO: 12.
[3] The antibody or the antigen-binding fragment thereof according to [1] or [2], comprising
(a) a heavy chain variable region (VH) comprising the amino acid sequence of SEQ ID NO: 3 or an amino acid sequence having 95% or higher identity to the amino acid sequence of SEQ ID NO: 3, and
(b) a light chain variable region (VL) comprising the amino acid sequence of SEQ ID NO: 9 or an amino acid sequence having 95% or higher identity to the amino acid sequence of SEQ ID NO: 9.
[4] The antibody or an antigen-binding fragment thereof according to any one of [1] to [3], comprising
(a) a heavy chain variable region (VH) comprising the amino acid sequence of SEQ ID NO: 3, and
(b) a light chain variable region (VL) comprising the amino acid sequence of SEQ ID NO: 9.
[5] The antibody or an antigen-binding fragment thereof according to any one of [1] to [4], wherein the antibody or the antigen-binding fragment thereof is of IgG1 (κ) class (subclass).
[6] The antibody or an antigen-binding fragment thereof according to any one of [1] to [5], wherein the antibody or the antigen-binding fragment thereof has a dissociation constant (KD value) of 1×10⁻⁹ M or lower against the Clostridium difficile toxin A.
[7] The antibody or an antigen-binding fragment thereof according to any one of [1] to [5], wherein the antibody or the antigen-binding fragment thereof has a neutralizing activity (EC50) of 0.05 μg/mL (approximately 0.33 nM) or lower against the Clostridium difficile toxin A in measurement using a human lung fibroblast IMR-90.
[8] An antibody capable of specifically binding to Clostridium difficile toxin B protein and neutralizing its biological activity, or an antigen-binding fragment thereof, containing:
(i)
(a) heavy chain CDR1 comprising the amino acid sequence of SEQ ID NO: 16 or an amino acid sequence deviated from the amino acid sequence of SEQ ID NO: 16 by deletion, substitution, insertion, and/or addition mutations of one to several amino acid residues,
(b) heavy chain CDR2 comprising the amino acid sequence of SEQ ID NO: 17 or an amino acid sequence deviated from the amino acid sequence of SEQ ID NO: 17 by deletion, substitution, insertion, and/or addition mutations of one to several amino acid residues, and
(c) heavy chain CDR3 comprising the amino acid sequence of SEQ ID NO: 18 or an amino acid sequence deviated from the amino acid sequence of SEQ ID NO: 18 by deletion, substitution, insertion, and/or addition mutations of one to several amino acid residues; and
(ii)
(a) light chain CDR1 comprising the amino acid sequence of SEQ ID NO: 22 or an amino acid sequence deviated from the amino acid sequence of SEQ ID NO: 22 by deletion, substitution, insertion, and/or addition mutations of one to several amino acid residues,
(b) light chain CDR2 comprising the amino acid sequence of SEQ ID NO: 23 or an amino acid sequence deviated from the amino acid sequence of SEQ ID NO: 23 by deletion, substitution, insertion, and/or addition mutations of one to several amino acid residues, and
(c) light chain CDR3 comprising the amino acid sequence of SEQ ID NO: 24 or an amino acid sequence deviated from the amino acid sequence of SEQ ID NO: 24 by deletion, substitution, insertion, and/or addition mutations of one to several amino acid residues.
[9] The antibody or an antigen-binding fragment thereof according to [8], comprising:
(i)
(a) the amino acid sequence of heavy chain CDR1 comprising the amino acid sequence of SEQ ID NO: 16,
(b) the amino acid sequence of heavy chain CDR2 comprising the amino acid sequence of SEQ ID NO: 17, and
(c) the amino acid sequence of heavy chain CDR3 comprising the amino acid sequence of SEQ ID NO: 18; and
(ii)
(a) the amino acid sequence of light chain CDR1 comprising the amino acid sequence of SEQ ID NO: 22,
(b) the amino acid sequence of light chain CDR2 comprising the amino acid sequence of SEQ ID NO: 23, and
(c) the amino acid sequence of light chain CDR3 comprising the amino acid sequence of SEQ ID NO: 24.
[10] The antibody or an antigen-binding fragment thereof according to [8] or [9], comprising
(a) a heavy chain variable region (VH) comprising the amino acid sequence of SEQ ID NO: 15 or an amino acid sequence having 95% or higher identity to the amino acid sequence of SEQ ID NO: 15, and
(b) a light chain variable region (VL) comprising the amino acid sequence of SEQ ID NO: 21 or an amino acid sequence having 95% or higher identity to the amino acid sequence of SEQ ID NO: 21.
[11] The antibody or an antigen-binding fragment thereof according to any one of [8] to [10], comprising
(a) a heavy chain variable region (VH) comprising the amino acid sequence of SEQ ID NO: 15, and
(b) a light chain variable region (VL) comprising the amino acid sequence of SEQ ID NO: 21.
[12] The antibody or an antigen-binding fragment thereof according to any one of [8] to [11], wherein the antibody or the antigen-binding fragment thereof is of IgG1 (λ) class (subclass).
[13] The antibody or an antigen-binding fragment thereof according to any one of [8] to [12], wherein the antibody or the antigen-binding fragment thereof has a neutralizing activity (EC50) of 0.1 μg/mL (approximately 0.7 nM) or lower against the Clostridium difficile toxin B in measurement using a human lung fibroblast IMR-90.
[14] A pharmaceutical composition comprising an antibody or an antigen-binding fragment thereof according to any one of [1] to [13] and a pharmaceutically acceptable carrier.
[15] The pharmaceutical composition according to [14], wherein the pharmaceutical composition comprises
(a) a first antibody specifically binding to Clostridium difficile toxin A protein or an antigen-binding fragment thereof according to any one of [1] to [7], and
(b) a second antibody specifically binding to Clostridium difficile toxin B protein or an antigen-binding fragment thereof according to any one of [8] to [13].
[16] The pharmaceutical composition according to [15], wherein the first and second monoclonal antibodies or antigen-binding fragments thereof neutralize the Clostridium difficile toxin A and the Clostridium difficile toxin B, respectively, in vitro or in vivo.
[17] The pharmaceutical composition according to any one of [14] to [16], wherein the pharmaceutical composition is intended for the treatment of Clostridium difficile infection.
[18] An isolated nucleic acid encoding the amino acid sequence of an antibody or an antigen-binding fragment thereof according to any one of [1] to [7], an isolated nucleic acid comprising a nucleotide sequence of SEQ ID NO: 1 or 7, or an isolated nucleic acid hybridizing under high stringent conditions to any of these nucleic acids.
[19] A vector comprising an isolated nucleic acid according to [18] therein.
[20] A host cell comprising a recombinant expression vector according to [19] introduced thereinto.
[21] An isolated nucleic acid encoding the amino acid sequence of an antibody or an antigen-binding fragment thereof according to any one of [8] to [13], an isolated nucleic acid comprising a nucleotide sequence of SEQ ID NO: 13 or 19, or an isolated nucleic acid hybridizing under high stringent conditions to any of these nucleic acids.
[22] A vector comprising an isolated nucleic acid according to [21] therein.
[23] A host cell comprising a recombinant expression vector according to [22] introduced thereinto.

### Advantageous Effects of Invention

The anti-toxin A antibody and the anti-toxin B antibody of the present invention or the antigen-binding fragments thereof specifically bind to toxin A and toxin B, respectively, and cancel (neutralize) their biological activity. Thus, the anti-toxin A antibody and the anti-toxin B antibody of the present invention or the antigen-binding fragments thereof can be expected to have therapeutic or preventive effects on CDI. Also, in an embodiment, the anti-toxin A antibody and the anti-toxin B antibody of the present invention are human monoclonal antibodies and thus probably have low immunogenicity.

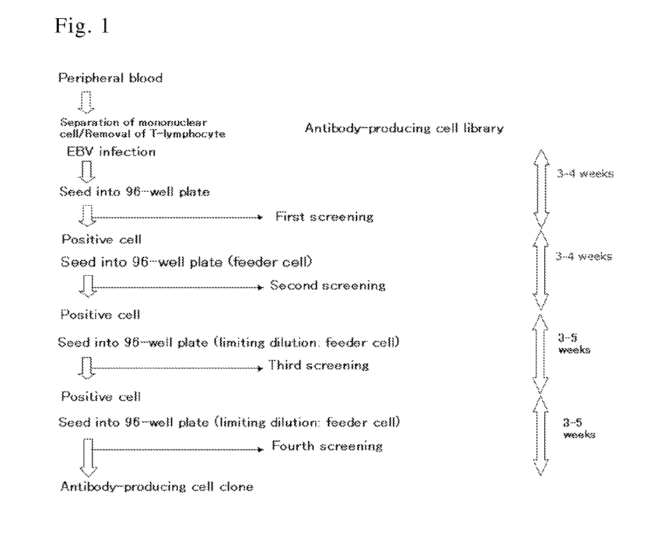

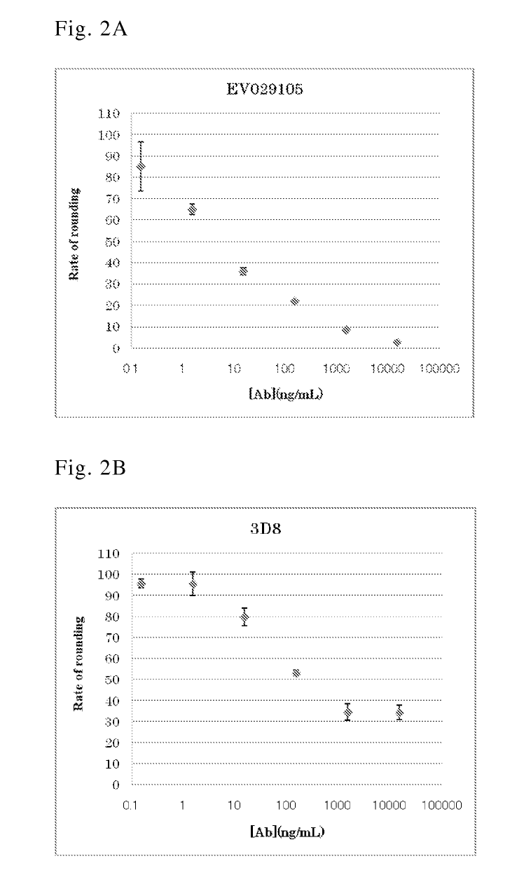

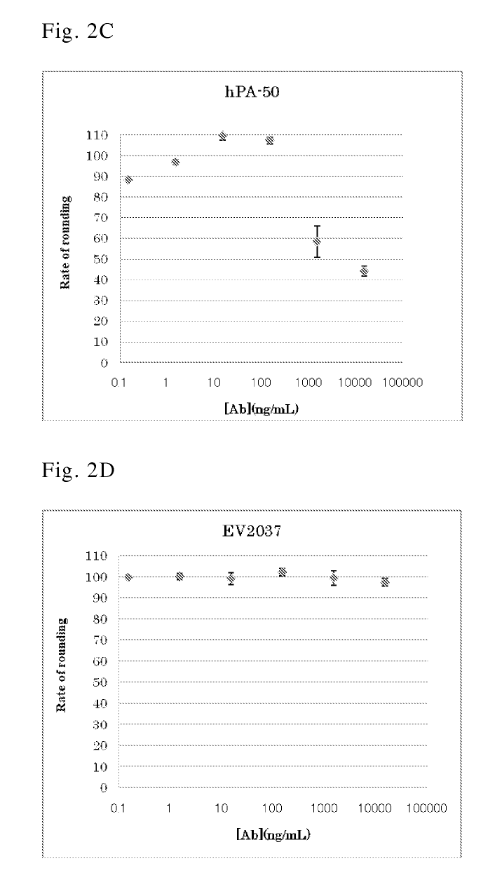

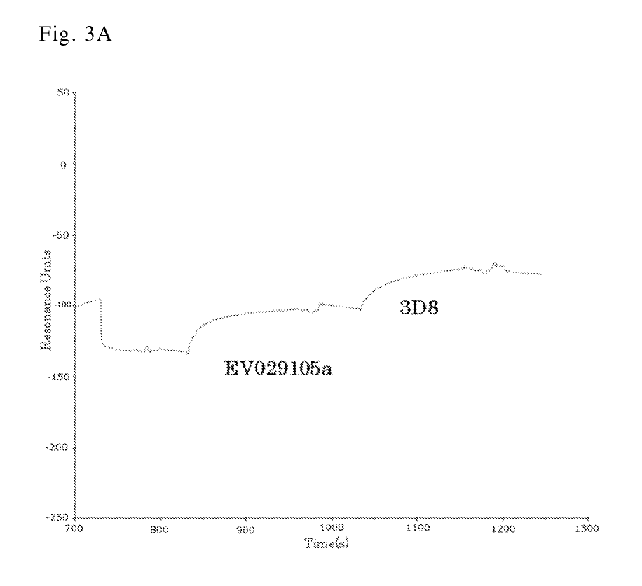

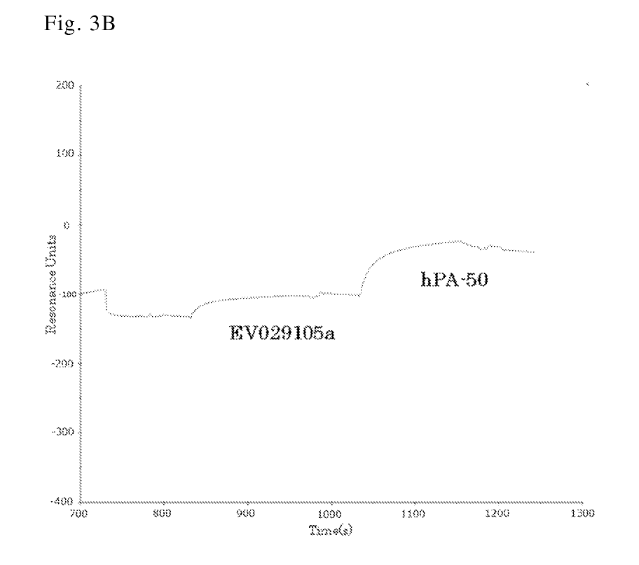

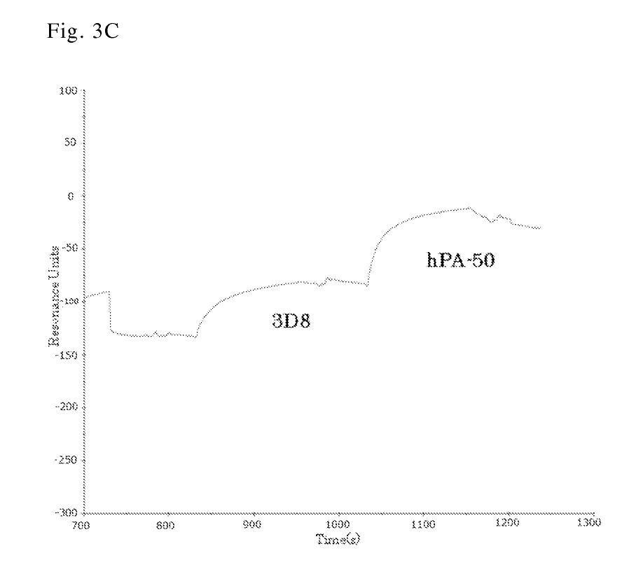

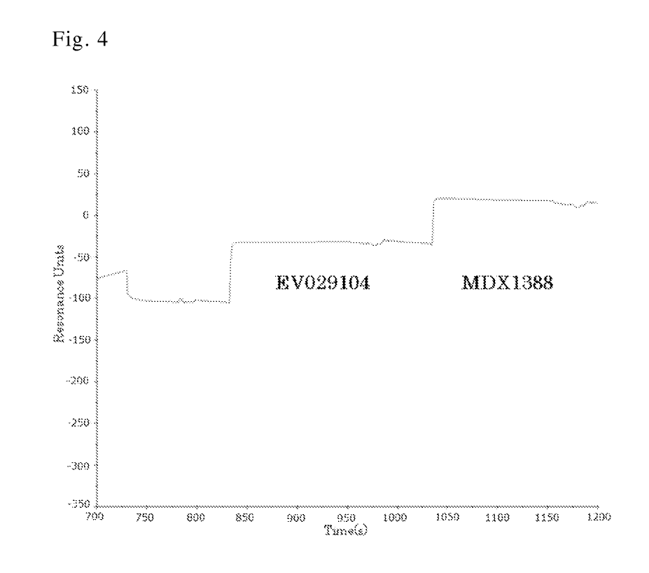

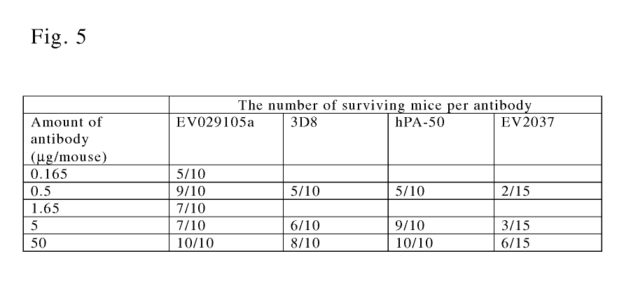

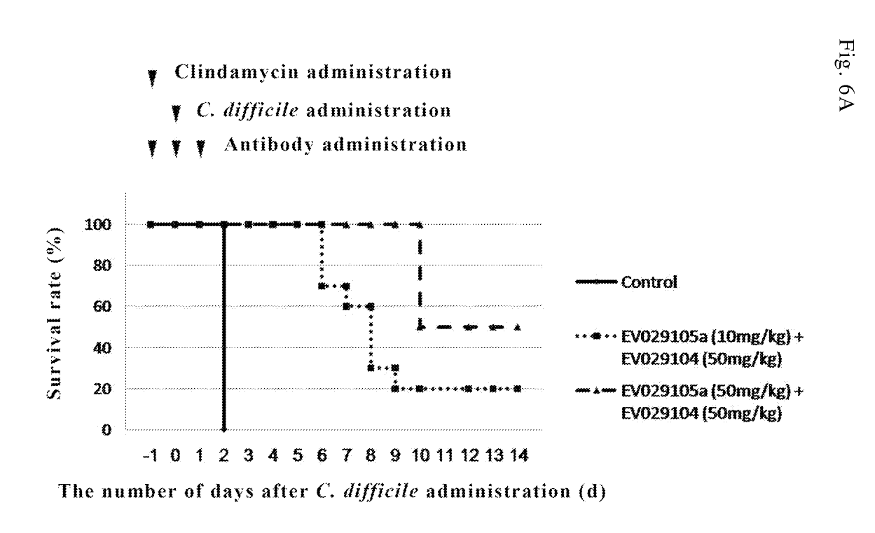

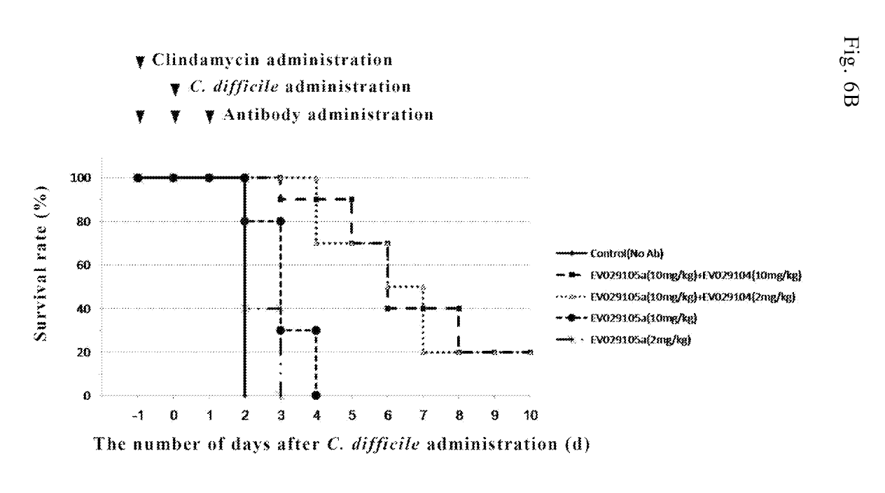

### BRIEF DESCRIPTION OF DRAWINGS

[FIG. 1] FIG. 1 is a diagram showing, in the form of flow chart, typical procedures of separating an antibody-producing cell clone producing the anti-C. difficile toxin antibody according to the present invention.

[FIG. 2A] FIGS. 2A to 2D are a set of graphs showing results of in vitro neutralization assay carried out in IMR-90 cells in the presence of an anti-toxin A monoclonal antibody. The number of inoculated cells was set to 3.5×10⁴ per well, and the concentration of toxin A was set to 20 ng/mL. For each antibody, the number of rounded cells with the addition of the toxin and without the addition of the antibody was defined as 100, while the number of rounded cells without the addition of the toxin and without the addition of the antibody was defined as 0. The cells were counted in three or more images taken for one field of view, and an average was calculated. FIG. 2A shows the neutralizing activity of EV029105a against toxin A. In the diagram, the ordinate indicates the rate of rounding in percentage, and the abscissa indicates antibody concentration (ng/mL).

[FIG. 2B] FIGS. 2A to 2D are a set of graphs showing results of in vitro neutralization assay carried out in IMR-90 cells in the presence of an anti-toxin A monoclonal antibody. The number of inoculated cells was set to 3.5×10⁴ per well, and the concentration of toxin A was set to 20 ng/mL. For each antibody, the number of rounded cells with the addition of the toxin and without the addition of the antibody was defined as 100, while the number of rounded cells without the addition of the toxin and without the addition of the antibody was defined as 0. The cells were counted in three or more images taken for one field of view, and an average was calculated. FIG. 2B shows the neutralizing activity of 3D8 against toxin A. In the diagram, the ordinate indicates the rate of rounding in percentage, and the abscissa indicates antibody concentration (ng/mL).

[FIG. 2C] FIGS. 2A to 2D are a set of graphs showing results of in vitro neutralization assay carried out in IMR-90 cells in the presence of an anti-toxin A monoclonal antibody. The number of inoculated cells was set to 3.5×10⁴ per well, and the concentration of toxin A was set to 20 ng/mL. For each antibody, the number of rounded cells with the addition of the toxin and without the addition of the antibody was defined as 100, while the number of rounded cells without the addition of the toxin and without the addition of the antibody was defined as 0. The cells were counted in three or more images taken for one field of view, and an average was calculated. FIG. 2C shows the neutralizing activity of hPA-50 against toxin A. In the diagram, the ordinate indicates the rate of rounding in percentage, and the abscissa indicates antibody concentration (ng/mL).

[FIG. 2D] FIGS. 2A to 2D are a set of graphs showing results of in vitro neutralization assay carried out in IMR-90 cells in the presence of an anti-toxin A monoclonal antibody. The number of inoculated cells was set to 3.5×10⁴ per well, and the concentration of toxin A was set to 20 ng/mL. For each antibody, the number of rounded cells with the addition of the toxin and without the addition of the antibody was defined as 100, while the number of rounded cells without the addition of the toxin and without the addition of the antibody was defined as 0. The cells were counted in three or more images taken for one field of view, and an average was calculated. FIG. 2D shows the neutralizing activity of EV2037 against toxin A. In the diagram, the ordinate indicates the rate of rounding in percentage, and the abscissa indicates antibody concentration (ng/mL).

[FIG. 3A] FIGS. 3A to 3C are a set of graphs showing the competitive binding of 3 anti-toxin A monoclonal antibodies (EV029105a, 3D8, and hPA-50) to toxin A using Biacore. To a sensor chip CAP, biotinylated toxin A was added, subsequently a first antibody was added, and finally a second antibody was added. In this competition assay, Biacore T200® apparatus was used. FIG. 3A shows results about the combined use of “EV029105a and 3D8” as the first and second antibodies. In the diagram, the ordinate indicates resonance units, and the abscissa indicates time (sec).

[FIG. 3B] FIGS. 3A to 3C are a set of graphs showing the competitive binding of 3 anti-toxin A monoclonal antibodies (EV029105a, 3D8, and hPA-50) to toxin A using Biacore. To a sensor chip CAP, biotinylated toxin A was added, subsequently a first antibody was added, and finally a second antibody was added. In this competition assay, Biacore T200® apparatus was used. FIG. 3B shows results about the combined use of “EV029105a and hPA-50” as the first and second antibodies. In the diagram, the ordinate indicates resonance units, and the abscissa indicates time (sec).

[FIG. 3C] FIGS. 3A to 3C are a set of graphs showing the competitive binding of 3 anti-toxin A monoclonal antibodies (EV029105a, 3D8, and hPA-50) to toxin A using Biacore. To a sensor chip CAP, biotinylated toxin A was added, subsequently a first antibody was added, and finally a second antibody was added. In this competition assay, Biacore T200® apparatus was used. FIG. 3C shows results about the combined use of “3D8 and hPA-50” as the first and second antibodies. In the diagram, the ordinate indicates resonance units, and the abscissa indicates time (sec).

[FIG. 4] FIG. 4 is a graph showing the binding of anti-toxin B monoclonal antibodies (EV029104 and MDX1388) to toxin B in the epitope analysis of each antibody by competition assay using Biacore. To a sensor chip CAP, biotinylated toxin B was added, subsequently the EV029104 antibody was added, and finally the MDX1388 antibody was added. In this competition assay, Biacore T200® apparatus was used.

[FIG. 5] FIG. 5 shows the protective effect of an anti-toxin A antibody against mouse lethality by toxin A. The number of surviving mice is indicated in numerator, and the number of mice in an administration group is indicated in denominator.

[FIG. 6A] FIG. 6A shows the protective effects of an anti-toxin A antibody and an anti-toxin B antibody against the lethality of hamsters by C. difficile. FIG. 6A shows results of comparing antibody administration groups with a group given only clindamycin without the administration of the antibody, wherein the antibody administration groups were set to two groups: a group given EV029105a and EV029104 both at 50 mg/kg per dose; and a group given EV029105a at 10 mg/kg and EV029104 at 50 mg/kg. The ordinate indicates the survival rate of hamsters, and the abscissa indicates the number of days after C. difficile administration.

[FIG. 6B] FIG. 6B shows the protective effects of an anti-toxin A antibody and an anti-toxin B antibody against the lethality of hamsters by C. difficile. FIG. 6B shows results of comparing antibody administration groups with a group given only clindamycin without the administration of the antibody, wherein the dose of the antibody was decreased and the antibody administration groups were set to four groups: a group given EV029105a at 10 mg/kg and EV029104 at 10 mg/kg per dose; a group given EV029105a at 10 mg/kg and EV029104 at 2 mg/kg; a group given only EV029105a at 10 mg/kg; and a group given only EV029105a at 2 mg/kg. The ordinate indicates the survival rate of hamsters, and the abscissa indicates the number of days after C. difficile administration.

### DESCRIPTION OF EMBODIMENTS

### 1. Definition

In the present specification, scientific terms and technical terms used in relation to the present invention have meanings generally understood by those skilled in the art. Words in the singular form shall be construed to include the plural and vice versa, unless the context otherwise requires. In general, nomenclatures used in relation to cells and techniques of tissue culture, molecular biology, immunology, microbiology, genetics, protein and nucleic acid chemistries, and hybridization described herein are well known in the art and generally used.

The terms used in the present invention are as defined below, unless otherwise specified.

1) Clostridium difficile (or C. difficile)

This bacterium is an obligately anaerobic gram-positive sporulating rod. The bacteria belonging to the genus Clostridium include, in addition to C. difficile, Clostridium tetani (tetanus bacillus), Clostridium botulinum, Clostridium perfringens, and the like. All of these bacteria are known to cause serious diseases. Until around the mid-1970s, the onset of pseudomembranous colitis (PMC) was recognized as being found particularly after use of some antibacterial drugs such as clindamycin and lincomycin. In recent years, however, this pseudomembranous colitis has been shown to be an inflammation in the large intestine that is manifested in response to toxins produced by C. difficile that has grown along with change in live bacterial flora in the intestine caused by the administration of antibiotics. C. difficile includes strains that produce no toxin, strains that produce low levels of toxins, and strains that produce high levels of toxins. Particularly, since 2003, a rise in the morbidity of C. difficile infection caused by the spread of hospital-acquired infection by a BUNAP1/027 strain or the like having high toxin productivity, and the resulting rise in death rate have been major social problems.

2) C. difficile Infection (CDI)

CDI is an infection by C. difficile and a series of following digestive system diseases caused by toxin production. The toxins produced by C. difficile act on the large intestinal epithelium to cause various degrees of damages from mild diarrhea to severe colitis. The most severe CDI cases are cases affected by pseudomembranous colitis (PMC) accompanied by terrible diarrhea, abdominal pain, and systemic signs such as fever, resulting in reportedly high fatality. Moderately severe cases are found to have terrible diarrhea, abdominal pain and tenderness, systemic signs (e.g., fever), and leukocytosis, etc., and also called antibiotic-associated colitis (AAC). Intestinal injury in AAC is less severe than that in PMC. AAC is free from endoscopic appearance characteristic of the colon in PMC and results in low death rate. Mild CDI cases are characterized by mild to moderate diarrhea associated with antibiotic administration. These mild cases are found to be free from an inflammation in the large intestine and systemic signs such as fever and also called antibiotic-associated diarrhea (AAD).

3) C. difficile Toxin

The toxins produced by C. difficile are mainly toxin A and toxin B proteins. The “toxin A” (or also referred to as “C. difficile toxin A”) is a protein encoded by a gene called tcdA on the C. difficile gene. Its amino acid sequence (SEQ ID NO: 25) has been registered under Accession No. P16154 with GenBank. The “toxin B” (or also referred to as “C. difficile toxin B”) is a protein encoded by a gene called tcdB on the C. difficile gene. Its amino acid sequence (SEQ ID NO: 26) has been registered under Accession No. Q46034 with GenBank. Both of these genes are located in a 19.6-kb gene region called Paloc (pathogenicity locus). In addition to tcdA and tcdB, genes, such as tcdC (negative regulator) and tcdD (positive regulator), which are involved in the control of the expression thereof, are present on this gene region.

The “toxin A” is a 308-kDa enterotoxin produced extracellularly by C. difficile. This protein is composed of 2710 amino acid residues and broadly constituted by 4 domains. Particularly, a repeat structure of amino acid sequences (CRD: C-terminal repetitive domain) is present in approximately ⅓ of the C-terminal side of the toxin A. This domain is considered to play a role in recognizing and binding to glycoproteins on cell surface and is also called receptor-binding domain (or RB domain). On the other hand, the amino-terminal region has glucosyltransferase activity targeting the Rho/Ras superfamily having GTPase activity and contains an enzyme domain (glucosyltransferase domain; also referred to as a GT domain) that glycosylates these targeted enzymes and blocks their phosphorylating ability. The activity is considered to destroy the integrity of actin polymerization and cytoskeleton of cells (Eichel-Streiber, Trends Microbiol., 1996 (4) p. 375-382). The N-terminal side of the intermediate moiety of the toxin protein contains a cysteine protease domain (also referred to as a CP domain) and is considered to participate in processing to separate the GT domain from the toxin protein. Also, the C-terminal side of the intermediate moiety contains a highly hydrophobic region probably involved in the passage of the toxin protein through the membrane and is called membrane insertion domain (Jank, Trends Microbiol., 2008 (16) p. 222-229; and Hussack, Toxins 2010 (2) p. 998-1018). For information, anti-toxin A antibodies 3D8 and hPA-50 have both been reported to bind to the RB domain present on the C-terminal side of the toxin A.

Likewise, the “toxin B” is a 269-kDa enterotoxin produced extracellularly by C. difficile. This protein is composed of 2366 amino acid residues, and its amino acid sequence has approximately 60% homology to the amino acid sequence of the “toxin A”. The protein is broadly constituted by 4 domains, as in the “toxin A”, and has an RB domain and a GT domain similar to those in the toxin A. An anti-toxin B antibody MDX1388 has been reported to bind to the RB domain present on the C-terminal side of the toxin B, while another anti-toxin B antibody hPA-41 has been reported to bind to the N-terminal side of the toxin B. Both of these toxins cause in vitro a cytopathic effect (CPE) on cultured cells such as Vero cells, human lung fibroblasts IMR-90, human colon epithelial cells T-84, and CHO-K1 cells, and the looping of intestinal tracts. It is also known that the toxins lead to death by in vivo exposure thereto or depending on the doses of the toxins in evaluation systems using models such as hamsters.

4) Anti-C. difficile Toxin Antibody

This antibody binds to a toxin protein (e.g., toxin A or toxin B) produced by C. difficile and is capable of binding to an epitope site, for example, a linear epitope or a conformational epitope, in the toxin protein, or a fragment of the toxin protein, etc.

In the present specification, the “anti-C. difficile toxin antibody”, the “antibody capable of neutralizing a C. difficile toxin”, or the “antibody capable of neutralizing the biological activity of a C. difficile toxin” refers to an antibody that inhibits the biological activity of the C. difficile toxin through binding to the C. difficile toxin.

The term “disease caused by a C. difficile toxin” used herein includes a disease for which the C. difficile toxin in a test subject affected by the disease has been shown or considered to be responsible for the pathophysiology of the disease or to contribute to deterioration in the disease, and also includes other diseases. Thus, the disease caused by a C. difficile toxin is a disease whose symptoms and/or progression are presumably alleviated by the inhibition of the biological activity of the C. difficile toxin. This disease corresponds to the aforementioned C. difficile infection (CDI).

The symptoms of CDI can be alleviated or cured, for example, by use of the aforementioned anti-C. difficile toxin antibody. Specifically, the symptoms of the disease can be alleviated or cured by raising the anti-C. difficile toxin antibody concentration in the body fluid of a test subject affected by the disease (e.g., raising the anti-C. difficile toxin antibody concentration in the serum, plasma, or synovial fluid of the test subject).

### 5) Antibody

The term “antibody” used herein refers to an immunoglobulin molecule composed of four polypeptide chains, i.e., two heavy (H) chains and two light (L) chains, linked via disulfide bonds. The monoclonal antibody according to the present invention is also composed of an immunoglobulin molecule comprising two heavy chains (H chains) and two light chains (L chains). Each H chain is composed of an H chain variable region (also referred to as “HCVR” or “VH”) and an H chain constant region (the H chain constant region is composed of three domains, which are also referred to as “CH1”, “CH2”, and “CH3”, respectively (collectively referred to as CH)). Each L chain is composed of an L chain variable region (also referred to as “LCVR” or “VL”) and an L chain constant region (the L chain constant region is composed of one domain, which is also referred to as “CL”). A region located before each constant region (also referred to as constant part, invariable region, or invariable part region) is called variable region (also referred to as variable part or variable part region).

Particularly, VH and VL are important because of their involvement in the binding specificity of the antibody for the antigen. Since the antibody interacts with its target antigen mainly through the amino acid residues of VH and VL, the amino acid sequences within the variable part regions differ more largely among individual antibodies than sequences without the variable part regions. VH and VL can be further subdivided into regions called framework regions (FRs) conserved among various antibodies, and hypervariable regions called complementarity-determining regions (CDRs). Each of VH and VL has three CDRs and four FRs, and these regions are arranged from the amino terminus to the carboxy terminus in the order of FR1, CDR1, FR2, CDR2, FR3, CDR3, and FR4.

### 6) “Antigen-Binding Fragment” of the Antibody (or Simply Referred to as “Antibody Fragment”)

The term “antigen-binding fragment” of the antibody (or simply referred to as an “antibody fragment”) used herein refers to one or more antibody fragments (e.g., VH) having the ability to specifically bind to the antigen (C. difficile toxin). The fragment also includes a peptide having the minimum amino acid sequence specifically binding to the antigen. Examples of binding moieties included in the term “antigen-binding fragment” of the antibody include (i) a Fab fragment, (ii) a F(ab′)2 fragment, (iii) an Fd fragment composed of VH and CH1 domains, (iv) an Fv fragment composed of VL and VH domains on a single arm of the antibody, (v) a dAb fragment composed of a VH domain (Ward et al., Nature 341: 544-546, 1989), (vi) isolated complementarity-determining regions (CDRs) having frameworks sufficient for the specific binding, and (vii) a bispecific antibody and (viii) a multispecific antibody composed of any combination of these fragments (i) to (vi). In the present specification, the term “antibody” used without discrimination includes not only whole antibodies but also these “antigen-binding fragments”.

### 7) Isotype

The isotype refers to the class (e.g., IgM or IgG1) of an antibody encoded by a heavy chain constant region gene. The antibody according to the present invention is preferably of IgG1 (κ) or IgG1 (λ) class (subclass).

### 8) Specific Binding

The term “specifically binding” or “specific binding” used herein refers to the recognition of the predetermined antigen so as to bind thereto.

Typically, the dissociation constant (KD value) of the anti-C. difficile toxin A antibody of the present invention against the toxin A is preferably 1×10⁻⁷ M or lower, more preferably 1×10⁻⁸ M or lower, further preferably 1×10⁻⁹ M or lower, most preferably 2×10⁻¹⁰ M or lower. The dissociation constant of the antibody against the toxin A can be measured using a method known in the art. For example, the dissociation constant can be measured against toxin A immobilized on a chip by use of a protein interaction analysis apparatus such as Biacore T200®.

### 9) Biological Activity of Antibody

The biological properties of the antibody or an antibody composition can be evaluated by testing the ability of the antibody to suppress in vitro the biological activity of the C. difficile toxin according to an assay method well known to those skilled in the art. The in vitro assay method includes a binding assay method such as ELISA, and a neutralization assay method, etc. In an in vivo evaluation system, its effectiveness for humans can be predicted from the ability of the anti-toxin antibody to protect an animal model against lethal attack by C. difficile. Specific examples of the animal model for predicting the effectiveness are described herein and further include an intestinal ligation model (WO2006/121422) and the like.

The terms such as “neutralization”, “inhibitory effect”, “inhibition”, “suppression”, “capable of inhibiting”, and “protection” used herein mean that biological activity attributed to the antigen (C. difficile toxin) is reduced by approximately 5 to 100%, preferably 10 to 100%, more preferably 20 to 100%, more preferably 30 to 100%, more preferably 40 to 100%, more preferably 50 to 100%, more preferably 60 to 100%, more preferably 70 to 100%, further preferably 80 to 100%.

The neutralizing ability of the anti-C. difficile toxin antibody can be evaluated using, for example, human lung fibroblasts IMR-90, by culturing, for 2 days, the cells exposed to toxin A and examining the resulting rate of rounding (cytopathic effect: CPE) of the cells in the presence of the anti-toxin A antibody.

The anti-C. difficile toxin A antibody of the present invention or the antigen-binding fragment thereof preferably has the ability to block approximately 50% cell rounding (EC50) at 1 μg/mL (approximately 7 nM) or lower, more preferably 0.1 μg/mL (approximately 0.7 nM) or lower, further preferably 0.05 μg/mL (approximately 0.33 nM) or lower, still further preferably 0.01 μg/mL (approximately 0.07 nM) or lower, most preferably approximately 0.005 μg/mL (approximately 0.033 nM) or lower, as the neutralizing activity against the toxin A using toxin A-exposed IMR-90 cells.

The anti-C. difficile toxin B antibody of the present invention or the antigen-binding fragment thereof preferably has the ability to block approximately 50% cell rounding (EC50) at 1 μg/mL (approximately 7 nM) or lower, more preferably 0.1 μg/mL (approximately 0.7 nM) or lower, as the neutralizing activity against the toxin B using toxin B-exposed IMR-90 cells.

### 10) Animal Model Experiment and Therapeutically Effective Amount

The ability of the antibody against measurable clinical parameters can be evaluated in an animal model system for predicting its effectiveness for humans. For example, the effectiveness for humans can be predicted from the ability of the anti-toxin antibody to protect a mouse against lethal attack by C. difficile. The amount of the anti-toxin antibody effective for the treatment of CDI or the “therapeutically effective amount” refers to the amount of the antibody effective for suppressing CDI in a subject when the antibody is administered at a single dose or plural doses to the subject. The therapeutically effective amount of the antibody or the antibody fragment may differ depending on factors such as the disease status, age, sex, and body weight of an individual, and the ability of the antibody or the antigen-binding fragment to induce the desired response in the individual. The therapeutically effective amount is an amount at which the therapeutically beneficial effect of the antibody or a moiety of the antibody exceeds its toxic effect or adverse effect.

The amount of the anti-toxin antibody effective for the prevention of the disease or the “preventively effective amount” of the antibody refers to the amount effective for hindering or delaying the onset or recurrence of CDI or for suppressing its symptoms when the antibody is administered at a single dose or plural doses to the subject. If longer prevention is desired, the antibody can be administered at an increased dose.

Example 12 of the present application demonstrated the in vivo protective effect of the anti-toxin A antibody against the lethal action of the toxin A on a mouse. In Example 12, EV029105a of the present invention intraperitoneally administered at 0.165 μg/head (approximately 8 μg/kg) was shown to have the activity of neutralizing 50% lethal action of toxin A administered at 200 ng/head. Also, EV029105a administered at 0.5 μg/head (approximately 25 μg/kg) was shown to be evidently superior in preventive effect to 3D8 or hPA-50 administered at the same dose thereas. Specifically, the anti-toxin A antibody according to the present invention or the antigen-binding fragment thereof includes an antibody having the activity of neutralizing 50% or more lethal action of toxin A administered at 200 ng/head when intraperitoneally administered at 0.165 μg/head (approximately 8 μg/kg or approximately 55 pmol/kg), and an antibody evidently superior in preventive effect to 3D8 or hPA-50 administered at the same dose thereas when administered at 0.5 μg/head (approximately 25 μg/kg or approximately 170 pmol/kg).

Furthermore, Example 13 demonstrated the remarkable protective effects of the anti-toxin A antibody and the anti-toxin B antibody against the lethal action of C. difficile on a Syrian golden hamster. Particularly, as shown in FIG. 6B, the combined administration of EV029105a and EV029104 was shown to have a very remarkable and dose-dependent protective effect against CDI, even as compared with the administration of EV029105a alone. Specifically, in one aspect, the present invention relates to a combined administration method using the anti-toxin A antibody or the antigen-binding fragment thereof and the anti-toxin B antibody or the antigen-binding fragment thereof. In this method, the dose of the first monoclonal antibody or the antigen-binding fragment thereof against the toxin A is preferably 1 mg/kg (approximately 6.7 nmol/kg) or larger, more preferably 10 mg/kg (approximately 67 nmol/kg) or larger, for example, when EV029105a is administered at a single dose or plural doses. In addition, the dose of the second monoclonal antibody or the antigen-binding fragment thereof used in combination therewith against the toxin B is preferably 1 mg/kg (approximately 6.7 nmol/kg) or larger, more preferably 10 mg/kg (approximately 67 nmol/kg) or larger, for example, when EV029104 is administered at a single dose or plural doses. However, the ranges of such doses are given merely for illustrative purposes and are not intended to limit the scope of the present invention. Depending on the type of a composition, one dose may be continuously administered using an infusion pump or the like, or divided doses may be administered at a given interval. It is well known that various protocols are possible as to doses, dosing schedules, modes of administration, and administration sites depending on the pathological conditions of a test subject, etc. The first monoclonal antibody or the antigen-binding fragment thereof is not limited to one type selected from the antibody group, and plural types may be selected. Likewise, one type of second monoclonal antibody or antigen-binding fragment thereof may be selected from the antibody group, or plural types may be selected depending on results of combination.

11) Procedure for C. difficile Infection (CDI)

The procedure for CDI means that the anti-toxin antibodies of the present invention or the antigen-binding fragments thereof are administered each alone or in combination, or together with a drug (e.g., an antibody drug or an antibiotic) other than the present invention, in order to treat or prevent CDI. The subject may be a patient infected by C. difficile or having the symptoms (e.g., diarrhea, colitis, and abdominal pain) of CDI or a predisposition of CDI (e.g., being treated with antibiotics, or having a history of CDI and a risk of recurrence of the disease). The procedure can be to treat, cure, alleviate, reduce, change, repair, ameliorate, lessen, or improve the symptoms of a disease associated with the infection or a predisposition of the disease, or to influence any of these procedures.

### 12) Substantially Identical

In relation to the term “substantially identical” used herein, the deletion, substitution, insertion, or addition of one or more amino acid residues in the amino acid sequence of the antibody of the present invention, or the combination of any two or more of these means that one or more amino acid residues are deleted, substituted, inserted, or added at one or more arbitrary positions in the same amino acid sequence, and two or more of the deletion, the substitution, the insertion, and the addition may occur at the same time.

Amino acids constituting proteins in the natural world can be grouped according to the properties of their side chains and can be classified into amino acid groups having similar properties, for example, groups of aromatic amino acids (tyrosine, phenylalanine, and tryptophan), basic amino acids (lysine, arginine, and histidine), acidic amino acids (aspartic acid and glutamic acid), neutral amino acids (serine, threonine, asparagine, and glutamine), hydrocarbon chain-containing amino acids (alanine, valine, leucine, isoleucine, and proline), and others (glycine, methionine, and cysteine).

As an example, amino acid residues that can be mutually substituted, also including nonnatural amino acids, may be grouped as follows, and the amino acid residues included in the same group can be mutually substituted: group A: leucine, isoleucine, norleucine, valine, norvaline, alanine, 2-aminobutanoic acid, methionine, o-methylserine, t-butylglycine, t-butylalanine, and cyclohexylalanine; group B: aspartic acid, glutamic acid, isoaspartic acid, isoglutamic acid, 2-aminoadipic acid, and 2-aminosuberic acid; group C: asparagine and glutamine; group D: lysine, arginine, ornithine, 2,4-diaminobutanoic acid, and 2,3-diaminopropionic acid; group E: proline, 3-hydroxyproline, and 4-hydroxyproline; group F: serine, threonine, and homoserine; and group G: phenylalanine, tyrosine, and tryptophan.

The identity of an amino acid sequence or a nucleotide sequence can be determined using the Karlin-Altschul algorithm BLAST (Proc. Natl. Acad. Sci. USA 872264-2268, 1990; and Proc Natl Acad Sci USA 90: 5873, 1993). A program called BLASTN or BLASTX based on the BLAST algorithm has been developed (Altschul S F, et al., J Mol Biol 215: 403, 1990). In the case of analyzing a nucleotide sequence using BLASTN, parameters are set to, for example, score=100 and wordlength=12. In the case of analyzing an amino acid sequence using BLASTX, parameters are set to, for example, score=50 and wordlength=3. In the case of using the BLAST and Gapped BLAST programs, default parameters of each program are used.

### 2. Antibody of the Present Invention or Antigen-Binding Fragment Thereof

### 1) Anti-Toxin a Antibody of the Present Invention or Antigen-Binding Fragment Thereof

In one aspect, the present invention provides an antibody capable of specifically binding to toxin A protein produced by C. difficile and neutralizing its biological activity, or an antigen-binding fragment thereof (hereinafter, referred to as the antibody of the present invention). In a more specific embodiment, the antibody of the present invention is an antibody capable of recognizing and binding to an epitope different from that for the anti-toxin A antibodies 3D8 and hPA-50, in the RB domain of toxin A and neutralizing the biological activity of the protein, or an antigen-binding fragment thereof.

In one embodiment, examples of the anti-toxin A antibody of the present invention include an antibody capable of specifically binding to toxin A protein produced by C. difficile and neutralizing its biological activity, wherein the antibody has the amino acid sequences of SEQ ID NOs: 4, 5, and 6 as the amino acid sequences of heavy chains CDR1, CDR2, and CDR3, respectively, and has the amino acid sequences of SEQ ID NOs: 10, 11, and 12 as the amino acid sequences of light chains CDR1, CDR2, and CDR3, respectively.

In another embodiment, examples of the anti-toxin A antibody of the present invention include an antibody substantially identical to the antibody of the preceding embodiment. Such an antibody includes an antibody capable of specifically binding to toxin A protein produced by C. difficile and neutralizing its biological activity, wherein the antibody has heavy chains CDR1 to CDR3 and light chains CDR1 to CDR3 composed of amino acid sequences substantially identical to SEQ ID NOs: 4, 5, and 6 and SEQ ID NOs: 10, 11, and 12, as the amino acid sequences of heavy chains CDR1, CDR2, and CDR3 and light chains CDR1, CDR2, and CDR3. Specifically, such an antibody has the neutralizing activity and can have, as heavy chain and light chain CDR sequences, amino acid sequences deviated from the amino acid sequences of SEQ ID NOs: 4, 5, and 6 and SEQ ID NOs: 10, 11, and 12 by deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues in each sequence, or the combination of two or more of these mutations. Its amino acid sequence except for the CDRs is not particularly limited, and a so-called CDR-grafted antibody having an amino acid sequence, except for CDRs, derived from another antibody, particularly, an antibody of different species is also encompassed by the antibody of the present invention. Of these antibodies, an antibody is preferred in which the amino acid sequence other than CDRs is also derived from a human. If necessary, its framework region (FR) may have the deletion, substitution, insertion, or addition of one or several (specific numbers are the same as above) amino acid residues, or the combination of two or more of these mutations.

In a further alternative embodiment, examples of the anti-toxin A antibody of the present invention include an antibody capable of specifically binding to toxin A protein produced by C. difficile and neutralizing its biological activity, comprising (a) a heavy chain variable region (VH) represented by the amino acid sequence of SEQ ID NO: 3; an amino acid sequence deviated from the amino acid sequence of SEQ ID NO: 3 by deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 95% or higher (preferably 96% or higher, 97% or higher, 98% or higher, 99% or higher, or 99.5% or higher) identity to the amino acid sequence of SEQ ID NO: 3, and (b) a light chain variable region (VL) represented by the amino acid sequence of SEQ ID NO: 9; an amino acid sequence deviated from the amino acid sequence of SEQ ID NO: 9 by deletion, substitution, insertion, or addition of one to several (specific numbers are the same as above) amino acid residues, or the combination of two or more of these mutations; or an amino acid sequence having 95% or higher (specific % is the same as above) identity to the amino acid sequence of SEQ ID NO: 9.

### 2) Anti-Toxin B Antibody of the Present Invention or Antigen-Binding Fragment Thereof

In one aspect, the present invention also provides an antibody capable of specifically binding to toxin B protein produced by Clostridium difficile and neutralizing its biological activity, or an antigen-binding fragment thereof. In a more specific embodiment, the present invention provides an antibody capable of specifically binding to an epitope different from that for the anti-toxin B antibodies MDX1388 and hPA-41, in the RB domain on the C-terminal side and neutralizing the biological activity of the protein, or an antigen-binding fragment thereof.

In one embodiment, examples of the anti-toxin B antibody of the present invention include an antibody capable of specifically binding to toxin B protein produced by C. difficile and neutralizing its biological activity, wherein the antibody has the amino acid sequences of SEQ ID NOs: 16, 17, and 18 as the amino acid sequences of heavy chains CDR1, CDR2, and CDR3, respectively, and has the amino acid sequences of SEQ ID NOs: 22, 23, and 24 as the amino acid sequences of light chains CDR1, CDR2, and CDR3, respectively.

In another embodiment, examples of the anti-toxin B antibody of the present invention include an antibody substantially identical to the antibody of the preceding embodiment. Such an antibody includes an antibody capable of specifically binding to toxin B protein produced by C. difficile and neutralizing its biological activity, wherein the antibody has heavy chains CDR1 to CDR3 and light chain CDR1 to CDR3 composed of amino acid sequences substantially identical to SEQ ID NOs: 16, 17, and 18 and SEQ ID NOs: 22, 23, and 24, as the amino acid sequences of heavy chains CDR1, CDR2, and CDR3 and light chains CDR1, CDR2, and CDR3. Specifically, such an antibody has the neutralizing activity and can have, as heavy chain and light chain CDR sequences, amino acid sequences deviated from the amino acid sequences of SEQ ID NOs: 16, 17, and 18 and SEQ ID NOs: 22, 23, and 24 by deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues in each sequence, or the combination of two or more of these mutations. The amino acid sequence except for the CDRs of the anti-toxin B antibody is not particularly limited, and a so-called CDR-grafted antibody having an amino acid sequence, except for CDRs, derived from another antibody, particularly, an antibody of different species is also encompassed by the antibody of the present invention. Of these antibodies, an antibody is preferred in which the amino acid sequence other than CDRs is also derived from a human. If necessary, its framework region (FR) may have the deletion, substitution, insertion, or addition of one or several (specific numbers are the same as above) amino acid residues, or the combination of two or more of these mutations.

In a further alternative embodiment, examples of the anti-toxin B antibody of the present invention include an antibody capable of specifically binding to toxin B protein produced by C. difficile and neutralizing its biological activity, comprising (a) a heavy chain variable region (VH) represented by the amino acid sequence of SEQ ID NO: 15; an amino acid sequence deviated from the amino acid sequence of SEQ ID NO: 15 by deletion, substitution, insertion, or addition of one to several (specifically, 1 to 9, 1 to 8, 1 to 7, 1 to 6, 1 to 5, 1 to 4, 1 to 3, 1 to 2, or 1) amino acid residues, or the combination of two or more of these mutations; or an amino acid sequence having 95% or higher (preferably 96% or higher, 97% or higher, 98% or higher, 99% or higher, or 99.5% or higher) identity to the amino acid sequence of SEQ ID NO: 15, and (b) a light chain variable region (VL) represented by the amino acid sequence of SEQ ID NO: 21; an amino acid sequence deviated from the amino acid sequence of SEQ ID NO: 21 by deletion, substitution, insertion, or addition of one to several (specific numbers are the same as above) amino acid residues, or the combination of two or more of these mutations; or an amino acid sequence having 95% or higher (specific % is the same as above) identity to the amino acid sequence of SEQ ID NO: 21.

A method known in the art can be used as a method for preparing these antibodies (Riechmann L, et al., Reshaping human antibodies for therapy. Nature, 332:323-327, 1988). In the present invention, a completely human antibody is preferred, as a matter of course.

### 3. Nucleic Acid Encoding Antibody of the Present Invention, Etc.

In another aspect, the present invention provides a nucleic acid (nucleotide) encoding the anti-toxin A monoclonal antibody capable of specifically binding to toxin A and neutralizing its biological activity, or the antigen-binding fragment thereof. Examples of such a nucleic acid include a nucleic acid encoding an amino acid sequence selected from the group consisting of SEQ ID NOs: 2 to 6 and 8 to 12, and an isolated nucleic acid having high identity to the nucleic acid. In this context, the term “having high identity” means sequence identity to an extent that permits hybridization under high stringent conditions to the predetermined nucleic acid sequence and means having, for example, 60%, 70%, 80%, 90%, or 95% or higher identity. The present invention provides an isolated nucleic acid selected from nucleic acids hybridizing thereto under high stringent conditions. Preferably, the nucleic acid is DNA or RNA, more preferably DNA.

The present invention includes a nucleic acid (nucleotide) encoding the anti-toxin B monoclonal antibody capable of specifically binding to C. difficile toxin B and neutralizing its biological activity, or the antigen-binding fragment thereof and also includes a nucleic acid encoding an amino acid sequence selected from the group consisting of SEQ ID NOs: 15 to 18 and 20 to 24, and an isolated nucleic acid having high identity to the nucleic acid. In this context, the term “having high identity” means sequence identity to an extent that permits hybridization under high stringent conditions to the predetermined nucleic acid sequence and means having, for example, 60%, 70%, 80%, 90%, or 95% or higher identity. The present invention provides an isolated nucleic acid selected from nucleic acids hybridizing thereto under high stringent conditions. Preferably, the nucleic acid is DNA or RNA, more preferably DNA.

The “high stringent conditions” are conditions involving, for example, 5×SSC, 5×Denhardt's solution, 0.5% SDS, 50% formamide, and 50° C. (see e.g., J. Sambrook et al., Molecular Cloning, A Laboratory Manual 2nd ed., Cold Spring Harbor Laboratory Press (1989), particularly, 11.45 “Conditions for Hybridization of Oligonucleotide Probes”). Under these conditions, it can be expected that a polynucleotide (e.g., DNA) having high identity is more efficiently obtained at a higher temperature. However, possible factors that influence the stringency of hybridization are a plurality of factors such as temperatures, probe concentrations, probe lengths, ionic strengths, times, and salt concentrations. Those skilled in the art can achieve similar stringency by appropriately selecting these factors.

The nucleic acid hybridizing under high stringent conditions includes a nucleic acid having, for example, 70% or higher, 80% or higher, 90% or higher, 95% or higher, 97% or higher, or 99% or higher identity to the nucleic acid encoding the amino acid sequence. The identity of a nucleotide sequence can be determined by use of the aforementioned identity search algorithm or the like (Proc. Natl. Acad. Sci. USA 872264-2268, 1990; and Proc Natl Acad Sci USA 90: 5873, 1993).

The nucleic acid preferred for the anti-toxin A antibody in the present invention is a nucleic acid having nucleotide sequences respectively encoding the amino acid sequences of SEQ ID NOs: 3 and 9, more preferably a nucleic acid having nucleotide sequences respectively encoding the amino acid sequences of SEQ ID NOs: 2 and 8 except for signal sequence moieties. The signal sequence moieties in SEQ ID NOs: 2 and 8 refer to amino acid sequences described to the left side of the moieties corresponding to the N-terminal amino acids in their respective variable region sequences (SEQ ID NOs: 3 and 9).

The nucleic acid is further preferably a nucleic acid comprising both of the nucleotide sequences of SEQ ID NOs: 1 and 7.

The nucleic acid preferred for the anti-toxin B antibody in the present invention is a nucleic acid having nucleotide sequences respectively encoding the amino acid sequences of SEQ ID NOs: 15 and 21, more preferably a nucleic acid having nucleotide sequences respectively encoding the amino acid sequences of SEQ ID NOs: 14 and 20 except for signal sequence moieties. The signal sequence moieties in SEQ ID NOs: 14 and 20 refer to amino acid sequences described to the left side of the moieties corresponding to the N-terminal amino acids in their respective variable region sequences (SEQ ID NOs: 15 and 21).

The nucleic acid is further preferably a nucleic acid comprising both of the nucleotide sequences of SEQ ID NOs: 13 and 19.

### 4. Vector, Host Cell, and Antibody Preparation Method of the Present Invention

The present invention also relates to a vector comprising the nucleic acid incorporated therein and a host cell comprising the vector introduced thereinto, and a method for preparing the antibody using the vector and the host cell.

The antibody of the present invention can also be prepared as a recombinant human antibody by use of a method known in the art (see e.g., Nature, 312: 643, 1984; and Nature, 321: 522, 1986). The antibody of the present invention can be prepared, for example, by culturing the host cell having the vector of the present invention incorporated therein and purifying the produced antibody from the culture supernatant or the like. More specifically, VH- and VL-encoding cDNAs can be respectively inserted to expression vectors for animal cells containing human antibody CH- and/or human antibody CL-encoding genes prepared from the same cell or another human cell to construct human antibody expression vectors, which are transferred to animal cells so that the antibody is produced by expression.

The vector to which the nucleic acid encoding VH or VL of the antibody of the present invention is incorporated is not necessarily limited and is preferably a vector that is routinely used for the expression of protein genes or the like and is particularly compatible with the expression of antibody genes, or a vector for high expression. Preferred examples thereof include vectors containing EF promoter and/or CMV enhancer. Usually, expression vectors respectively having VH- or VL-encoding nucleic acids incorporated therein are prepared, and host cells are cotransfected with the expression vectors. Alternatively, these nucleic acids may be incorporated in a single expression vector.

The host cell to which the expression vector is transferred is not necessarily limited and is preferably a cell that is routinely used for the expression of protein genes or the like and is particularly compatible with the expression of antibody genes. Examples thereof include bacteria (E. coli, etc.), Actinomycetes, yeasts, insect cells (SF9, etc.), and mammalian cells (COS-1, CHO, myeloma cells, etc.).

For the industrial production of the antibody, a recombinant animal cell line, for example, a CHO cell line, stably highly producing the antibody is generally used. The preparation and cloning of such a recombinant cell line, gene amplification for high expression, and screening can be carried out by use of methods known in the art (see e.g., Omasa T.: J. Biosci. Bioeng., 94, 600-605, 2002).

The antibody of the present invention includes an antibody composed of two heavy chains and two light chains as well as an antigen-binding fragment of the antibody. The antigen-binding fragment includes, for example, Fab (fragment of antigen binding), Fab′, F(ab′)2, and single chain Fv (scFv) and disulfide stabilized Fv (dsFv) as active fragments of the antibody linked via a linker or the like. Examples of peptides comprising the active fragments of the antibody include peptides containing CDRs. These fragments can be produced by a method known in the art such as a method of treating the antibody of the present invention with an appropriate protease or a gene recombination technique.

The antibody can be purified by use of purification means known in the art such as salting out, gel filtration, ion-exchange chromatography, or affinity chromatography.

Alternatively, according to a recently developed phage display antibody technique which involves expressing a recombinant antibody on phage surface by use of a genetic engineering technique, human VH and VL genes may be artificially shuffled, and diversified scFv (single chain fragment of variable region) antibodies can be expressed as phage-fused proteins to obtain specific antibodies. Any specific antibody or antigen-binding fragment thereof prepared by use of this technique with reference to the amino acid sequences of SEQ ID NOs: 2 to 6, 8 to 12, 14 to 18, and 20 to 24 described herein is included in the technical scope of the present invention. In addition, a recombinant antibody having high neutralizing activity or excellent thermal stability modified by the application of a humanized antibody preparation technique on the basis of the CDR sequence information described above (see e.g., WO2007/139164) is also included in the scope of the present invention.

Alternatively, an antibody or the like prepared by a recently developed technique of drastically improving the ADCC activity of an antibody by the modification of the sugar chain moiety of the antibody, for example, an antibody obtained by the application of Potelligent technique to the antibody of the present invention (see Niwa R., et al, Clin. Cancer Res., 10, 6248-6255 (2004)), and an antibody or the like prepared by a technique of improving CDC activity, for example, an antibody obtained by the application of Complegent technique to the antibody of the present invention (see Kanda S., et al, Glycobiology, 17, 104-118 (2007)) are also included in the technical scope of the present invention. In addition, an antibody obtained by an antibody modification technique intended to maintain the in vivo concentration of the antibody by the partial substitution of an Fc region (see e.g., WO2007/114319) is also included in the technical scope of the present invention.

Furthermore, any antibody or antigen-binding fragment thereof obtained by the application of a partial Fc region substitution technique (see WO2006/071877) performed in order to impart protease resistance ability to the antibody and renders the antibody orally administrable is included in the technical scope of the present invention.

As an antibody preparation approach, a polyclonal antibody or a monoclonal antibody is usually obtained by use of a laboratory animal such as a mouse, a rabbit, or a goat. Since the antibody thus obtained has a sequence characteristic of the animal species used, the antibody administered directly to a human may be recognized as foreign matter by the human immune system to cause human anti-animal antibody response (i.e., to yield an antibody against the antibody).

The anti-C. difficile toxin monoclonal antibody of the present invention or the antigen-binding fragment thereof can be obtained from antibody-producing cells derived from the blood of a healthy person or the like. In this case, the antibody is a completely human antibody. This completely human antibody probably has low immunogenicity even if administered as an antibody drug to a human body.

Particularly, the anti-C. difficile toxin A monoclonal antibody of the present invention has higher neutralizing ability than that of the conventional anti-C. difficile toxin A monoclonal antibodies and as such, can be expected to produce equivalent therapeutic effects at a lower dose.

### 5. Pharmaceutical Composition Containing Antibody of the Present Invention

The present invention further provides a pharmaceutical composition for the prevention or treatment of a disease involving a C. difficile toxin, comprising the antibody or the antigen-binding site thereof and a pharmaceutically acceptable carrier.

Particularly, the anti-C. difficile toxin A antibody of the present invention or the antigen-binding fragment thereof has higher neutralizing ability against toxin A than that of the conventional anti-toxin A antibodies and as such, is useful as a preventive or therapeutic drug for a disease involving the C. difficile toxin. As shown in Example 13, the neutralizing antibody against toxin A can be used not only alone but in combination with the neutralizing antibody against toxin B in the treatment of CDI. The combined administration can further enhance preventive and therapeutic effects on CDI compared with the administration of each antibody alone. Specifically, the combined administration of a pharmaceutical composition comprising any one antibody selected from among the anti-toxin A antibodies of the present invention and the antigen-binding fragments thereof or a pharmaceutical composition comprising a plurality of antibodies selected from thereamong, and a pharmaceutical composition comprising any one antibody selected from among the anti-toxin B antibodies of the present invention and the antigen-binding fragments thereof or a pharmaceutical composition comprising a plurality of antibodies selected from thereamong can be expected to produce a remarkable protective effect against CDI.

The “pharmaceutically acceptable carrier” used herein includes any or every biologically compatible solvent, dispersion, coating, tonicity agent, and absorption-delaying agent, etc.

Examples of the pharmaceutically acceptable carrier include one or more carriers such as water, a salt solution, phosphate-buffered saline, dextrose, glycerol, and ethanol, and combinations thereof. For use as an injection or the like, the composition preferably contains a pH adjuster or a tonicity agent, for example, a sugar, a polyalcohol (mannitol, sorbitol, etc.), or sodium chloride. The pharmaceutically acceptable carrier can further include a small amount of an auxiliary substance that enhances the conservation or effectiveness of the antibody or a moiety of the antibody, such as a wetting agent, an emulsifier, an antiseptic, a buffer, or a stabilizer.

The composition of the present invention can be prepared in various dosage forms. Such a composition includes liquid, semisolid, and solid dosage forms, for example, solutions (e.g., injectable and transfusable solutions), dispersions, suspensions, tablets, capsules, troches, pills, powders, liposomes, and suppositories. Preferred forms differ depending on the intended mode of administration and a case to which treatment is applied. The composition is generally preferably in the form of an injectable or transfusable solution, as with, for example, compositions similar to those used for the passive immunization of humans with other antibodies. The preferred mode of administration is parenteral administration (e.g., intravenous, subcutaneous, intraperitoneal, or intramuscular administration). In a preferred embodiment, the antibody is administered through intravenous transfusion or intravenous injection. In another preferred embodiment, the antibody is administered through intramuscular injection or subcutaneous injection.

The antibody of the present invention or the antibody fragment can be incorporated into a pharmaceutical composition suitable for parenteral administration. For example, when the antibody or the antibody fragment is prepared as an injectable preparation, the concentration range of the preparation is preferably 0.1 to 200 mg/mL, more preferably 1 to 120 mg/mL, further more preferably 2 to 25 mg/mL.

Exemplary injectable preparations will be described below. However, the preparation of the present invention is not limited thereto as long as the preparation is preferred as an injection of the antibody drug of the present invention. The preparation can be constituted by, for example, a flint or amber vial, an ampule, or a prefilled syringe containing an active ingredient dissolved in a liquid or a freeze-dried active ingredient. The buffer can be L-histidine (1 to 50 mM) having a pH of 5.0 to 7.0 (optimally pH 6.0), optimally 5 to 10 mM L-histidine. Other appropriate buffers include, but are not limited to, sodium succinate, sodium citrate, sodium phosphate, and potassium phosphate. Sodium chloride can be used in order to change the osmotic pressure of a solution having a concentration of 0 to 300 mM (optimally 150 mM for the liquid dosage form,). The freeze-dried dosage form can contain a cryoprotectant, mainly, 0 to 10% (optimally 0.5 to 5.0%) sucrose. Other appropriate cryoprotectants include mannitol, trehalose, and lactose. The freeze-dried dosage form can contain an expander, mainly, 1 to 10% (optimally 2 to 4%) mannitol. In both of the liquid and freeze-dried dosage forms, a stabilizer, mainly, 1 to 50 mM (optimally 5 to 10 mM) L-methionine can be used. Other appropriate stabilizers include glycine, arginine, and polysorbate 80, etc. In the case of polysorbate 80, 0 to 0.05% (optimally 0.005 to 0.01%) can be contained therein. Other surfactants include, but are not limited to, polysorbate 20 and BRIJ surfactants.

In general, the pharmaceutical composition of the present invention must be sterile or stable under production and preservation conditions. This composition can be formulated as a solution, a microemulsion, a dispersion, a liposome, or other ordered structures suitable for high drug concentrations. The sterile injectable solution can be prepared by mixing a necessary amount of an active compound (i.e., the antibody or a moiety of the antibody), if necessary together with one or combination of the aforementioned components, into an appropriate solvent, followed by filtration sterilization. In general, the active compound is mixed with a sterile vehicle containing a basic dispersion medium and other necessary components selected from those listed above to prepare a dispersion. Preferred methods for preparing a sterile powder preparation for preparing the sterile injectable solution are the vacuum freeze drying and spray drying of the sterile filtrate thereof mentioned above. As a result, a composition comprising a powder of the active ingredient as well as arbitrary other desired components is obtained. The adequate flowability of the solution can be maintained, for example, by using coating with lecithin or the like, by maintaining a necessary particle size in the case of the dispersion, or by using a surfactant. The long-term absorption of the injectable composition can be achieved by means of absorption-delaying agent, for example, monostearate or gelatin, contained in the composition.

The present invention also relates to a kit comprising the anti-toxin antibody of the present invention or the antigen-binding site thereof. The kit may contain, for example, one or more additional components including other substances (pharmaceutically acceptable carriers) listed herein for preparing the pharmaceutical composition of the antibody, and an apparatus or other substances for administration to a subject. In this case, various combinations of pharmaceutical compositions of individual antibodies can be packaged together. The pharmaceutical compositions contained in the kit are constituted by, for example, pharmaceutical compositions respectively comprising any one or more selected from among the first monoclonal antibodies neutralizing toxin A or the antigen-binding fragments thereof, and any one or more selected from among the second monoclonal antibodies neutralizing toxin B or the antigen-binding fragments thereof. Preferably, the first antibody is EV029105a or an anti-toxin A antibody within the scope of the present invention having neutralizing activity equivalent to or higher than that of EV029105a, and the second antibody is EV029104 or an anti-toxin B antibody within the scope of the present invention having neutralizing activity equivalent to or higher than that of EV029104. Particularly, plural types of antibodies may be mixed, and the resulting preparation can be packaged in the kit as long as there is no problem associated with stability.

### 6. Approach of Obtaining Anti-C. difficile Toxin Antibody of the Present Invention and Antigen-Binding Fragment Thereof

Next, the approaches by which the anti-C. difficile toxin monoclonal antibody of the present invention and the antigen-binding fragment thereof were obtained will be described. However, the approaches of obtaining the antibody of the present invention, etc., are not limited by the description below. As mentioned above, changes or modifications usually performed in the art can be made therein, as a matter of course.

The anti-C. difficile toxin antibody of the present invention and the antigen-binding fragment thereof can be obtained by: separating a cell clone producing the antibody through various steps from the blood of a human; isolating cDNA from the obtained cell clone and amplifying the cDNA; transferring plasmids having the cDNA incorporated therein to producing cells; and culturing the obtained antibody-producing cells, followed by, for example, affinity purification from the supernatant.

1) Separation of Cell Clone Producing Completely Human Antibody Against C. difficile Toxin

B lymphocytes are separated from the blood of a human, and the growth of the B lymphocytes is induced. The method for inducing the growth is known in the art per se and can be carried out by, for example, a transformation method (D. Kozbor et al.) using “Epstein-Barr virus (EB virus)” (hereinafter, referred to as EBV), which is a factor triggering cancer.

Specifically, the B lymphocytes are infected by EBV to induce its growth. The cells that have grown are used as an antibody-producing cell library.

2) Recovery of Monoclonal Antibody from Antibody-Producing Cell Library

The method for recovering monoclonal antibodies from the cells that have grown by induction can be carried out by a well known method routinely used in the preparation of monoclonal antibodies.

The antibody-producing cell library is screened for a lymphocyte clone producing an antibody binding to C. difficile toxin A or/and C. difficile toxin B. From the obtained cell clone, cDNA is isolated and amplified. Plasmids having an insert of the cDNA are transferred to producing cells. The obtained antibody-producing cells are cultured, and the antibody is isolated from the culture supernatant. In order to separate the cell clone of interest from the antibody-producing cell library, a cell population (clone) producing the antibody binding to the C. difficile toxin can be selected by the appropriate combination of a limiting dilution culture method, a sorting method, and a cell microarray method.

The clone binding to the C. difficile toxin is preferably detected by use of ELISA with the C. difficile toxin as an antigen and ELISA using a labeled mouse anti-human IgG antibody.

The selected antibody-positive cell population can be cultured and repeatedly screened to obtain a cell population (clone) producing only the antibody of interest.

A flowchart representing these steps up to the separation of the antibody-producing cell clone is shown in FIG. 1.

### 3) Affinity Purification Using Protein A or G

For the purification of the anti-C. difficile toxin antibody, antibody-producing cells can be obtained by a gene recombination approach from the selected cells and allowed to grow in a roller bottle, a 2-L spinner flask, or a different culture system.

The obtained culture supernatant can be filtered, concentrated, and then subjected to affinity chromatography using protein A or protein G-Sepharose (GE Healthcare Japan Corp.) or the like to purify the protein. The buffer solution is replaced with PBS, and the concentration can be determined by OD280 or, preferably, nephelometer analysis. The isotype can be examined by a method specific for the isotype antigen. The anti-C. difficile toxin antibody thus obtained is a completely human antibody prepared from B lymphocytes sensitized in the human body and is therefore substantially unlike to cause immune response.

Another feature of this approach is that the EB virus having the activity of inducing the growth of B lymphocytes by infection is used in the preparation of the antibody-producing cell clone.

The EB virus method has the advantages that: a natural human antibody produced in a human body can be prepared; and an antibody having high affinity can be obtained. For example, a human antibody against a certain kind of virus (e.g., human CMV) has been found to have approximately 10 to 100 times higher affinity than that of an antibody prepared from an artificially immunized mouse. The B lymphocyte population that has grown by EB virus infection serves as a library of antibody-producing cells. From this library, a particular antibody-producing cell clone is separated, and antibody-producing cells can be obtained by a gene recombination method from the separated and selected cells and cultured to obtain a human antibody.

### EXAMPLES

Hereinafter, the present invention will be described further specifically with reference to Examples. However, the present invention is not intended to be limited by these Examples by any means. For the procedures used in these Examples, see Molecular Cloning: A Laboratory Manual (Third Edition) (Sambrook et al., Cold Spring Harbour Laboratory Press, 2001), unless otherwise specified.

### Example 1

### Separation of Cell Clone Producing Completely Human Antibody Against C. difficile Toxin a or Toxin B

A typical flowchart for the separation of an antibody-producing cell clone is shown in FIG. 1.

B lymphocytes were separated from anti-C. difficile toxin antibody-positive human peripheral blood and infected by EBV. The infected cells were inoculated to a 96-well plate, cultured for 3 to 4 weeks, and then screened for anti-C. difficile toxin antibodies in the culture supernatant. The screening was carried out by ELISA targeting antibodies against toxin A (SEQ ID NO: 25; GenBank Accession No. P16154) and toxin B (SEQ ID NO: 26; GenBank Accession No. Q46034) (Clin. Microbiol. Rev., 18, 247-263 (2005); and GlycoBiol., 17, 15-22 (2007)), which are primary exotoxins of C. difficile, and using a 96-well plate coated with toxin A or toxin B (both obtained from List Biological Laboratories Inc.). The cells in each well confirmed to contain the produced anti-C. difficile toxin antibodies were subjected to a limiting dilution culture method appropriately combined with a sorting method and a cell microarray method to separate a cell clone producing each antibody of interest.

### Example 2

### Confirmation of Antibody Isotype and Subclass

Each produced antibody was isotyped by ELISA using the culture supernatant of the separated antibody-producing cell clone (see reference: Curr Protoc Immunol. 2001 May; Chapter 2: Unit 2.2). This ELISA employed a 96-well plate coated with toxin A or toxin B to which each anti-toxin antibody was allowed to bind. Next, an antibody specific for each isotype and subclass was used as a secondary antibody. The isotype and subclass of the obtained anti-toxin A antibody or anti-toxin B antibody is shown in Table 1.

TABLE 1

| Antibody No. | Target antigen | Subclass |
|---|---|---|
| EV029105a | Toxin A | IgG1/κ |
| EV029104 | Toxin B | IgG1/λ |

### Example 3

### Cloning of cDNA Encoding Anti-C. difficile Toxin Antibody

The total RNA of the antibody-producing cells was reverse-transcribed using oligo-dT primers. The obtained cDNA was used as a template in the PCR amplification of each antibody gene. The primers used in PCR were designed on the basis of the database of cDNAs encoding human IgG antibody H and L chains. In order to amplify the full-length H chain cDNA and L chain cDNA, the 5′ primer has a translation initiation point, and the 3′ primer has a translation termination point.

### Example 4

### Determination of Amino Acid Sequence of Antibody Based on Nucleotide Sequence

The H chain and L chain cDNAs of each antibody amplified by PCR were inserted to plasmid vectors, and their nucleotide sequences were confirmed using an ABI sequencer. The signal sequence, H chain and L chain amino acid sequences, variable region amino acid sequences, and complementarity-determining region (CDR) amino acid sequences of the antibody were each determined from the obtained nucleotide sequences. In the CDR analysis, the method of Kabat (www.bioinf.org.uk: Dr. Andrew C.R. Martin's Group, Antibodies: General Information) was used.

SEQ ID NO of each sequence is described below.

SEQ ID NO: 1: the nucleotide sequence of the H chain (including the signal sequence) of EV029105a

SEQ ID NO: 2: the amino acid sequence of the H chain (including the signal sequence) of EV029105a

SEQ ID NO: 3: the amino acid sequence of the H chain variable region of EV029105a

SEQ ID NO: 4: the amino acid sequence of the H chain CDR1 region of EV029105a

SEQ ID NO: 5: the amino acid sequence of the H chain CDR2 region of EV029105a

SEQ ID NO: 6: the amino acid sequence of the H chain CDR3 region of EV029105a

SEQ ID NO: 7: the nucleotide sequence of the L chain (including the signal sequence) of EV029105a

SEQ ID NO: 8: the amino acid sequence of the L chain (including the signal sequence) of EV029105a

SEQ ID NO: 9: the amino acid sequence of the L chain variable region of EV029105a

SEQ ID NO: 10: the amino acid sequence of the L chain CDR1 region of EV029105a

SEQ ID NO: 11: the amino acid sequence of the L chain CDR2 region of EV029105a

SEQ ID NO: 12: the amino acid sequence of the L chain CDR3 region of EV029105a

SEQ ID NO: 13: the nucleotide sequence of the H chain (including the signal sequence) of EV029104

SEQ ID NO: 14: the amino acid sequence of the H chain (including the signal sequence) of EV029104

SEQ ID NO: 15: the amino acid sequence of the H chain variable region of EV029104

SEQ ID NO: 16: the amino acid sequence of the H chain CDR1 region of EV029104

SEQ ID NO: 17: the amino acid sequence of the H chain CDR2 region of EV029104

SEQ ID NO: 18: the amino acid sequence of the H chain CDR3 region of EV029104

SEQ ID NO: 19: the nucleotide sequence of the L chain (including the signal sequence) of EV029104

SEQ ID NO: 20: the amino acid sequence of the L chain (including the signal sequence) of EV029104

SEQ ID NO: 21: the amino acid sequence of the L chain variable region of EV029104

SEQ ID NO: 22: the amino acid sequence of the L chain CDR1 region of EV029104

SEQ ID NO: 23: the amino acid sequence of the L chain CDR2 region of EV029104

SEQ ID NO: 24: the amino acid sequence of the L chain CDR3 region of EV029104

### Example 5

### Confirmation that Obtained Antibody Gene Encoding Anti-C. difficile Toxin Antibody

The obtained H chain and L chain cDNAs were respectively inserted to expression vectors. CHO cells were cotransfected with the expression vectors for transient expression. The transfection was carried out using Lipofectamine (Invitrogen Corp.) and Plus reagent (Invitrogen Corp.) under conditions recommended by the manufacturer (Invitrogen catalog: Cat. No. 18324-111, Cat. No. 18324-012, or Cat. No. 18324-020). Two days later, the culture supernatant was recovered. By ELISA using an anti-human IgG antibody and a 96-well plate coated with toxin A or toxin B, it was confirmed that: the antibodies in the culture supernatants of EV029105a and EV029104 were human IgG antibodies; and EV029105a and EV029104 specifically bound to toxin A and toxin B, respectively.

### Example 6

### Production of Antibody Protein

CHO cells were transfected with the expression plasmid for each obtained anti-C. difficile toxin antibody. The transfection was carried out in the same way as above. The cells were cultured in the presence of a selection marker to obtain a CHO cell clone constitutively producing each antibody.

The CHO cells stably producing each antibody were cultured in a serum-free medium, and each culture supernatant was recovered. This culture supernatant was added to a protein A column and subjected to affinity purification to obtain a purified antibody. The column used was a prepack column of HiTrap rProtein A FF (GE Healthcare Japan Corp.). The purification conditions were set to conditions recommended by the column manufacturer. After the purification, the binding activity of each antibody against toxin A or toxin B was confirmed by ELISA. Also, the presence of an antibody H chain of approximately 50 kDa and an antibody L chain of approximately 25 kDa was confirmed in each antibody by SDS-PAGE.

### Example 7

### Neutralizing Activity of Anti-Toxin A Antibody

Each antibody was tested for its neutralizing activity against toxin A in vitro (Babcock et al., Infect. Immun 74: 6339-6347 (2006)). Toxin A was reacted in the presence of varying concentrations of the toxin A-specific monoclonal antibody and then added to cells to evaluate the ability to block the rounding of the toxin A-exposed cells. Human lung fibroblasts IMR-90 were used as the target cells of toxin A. For the activity evaluation, the rate of rounding (%) was determined by the visual observation of the cells and indicated as a cytopathic effect (CPE). The anti-toxin A antibodies used in this activity evaluation were EV029105a and two already publicly available anti-toxin A antibodies 3D8 (WO2006/121422) and hPA-50 (WO2011/130650). The antibodies 3D8 and hPA-50 were both prepared on the basis of their reported nucleotide sequences. A human monoclonal antibody (EV2037; WO2010/114105) specific for human cytomegalovirus (HCMV) was used as a negative control. The results of these experiments are shown in FIGS. 2A to 2D. These diagrams showed that all of the antibodies except for EV2037 suppress the rounding of cells by toxin A. The neutralizing activity (EC50 value) of these anti-toxin A antibodies against the cytotoxicity of toxin A to the IMR-90 cells is shown in Table 2. Among these three anti-toxin A antibodies, an antibody having high neutralizing activity of 100 ng/mL or lower was only EV029105a.

TABLE 2

| Antibody No. | EC50 (ng/mL) |
|---|---|
| EV029105a | 4.92 ± 0.78 (0.033 nM) |
| 3D8 | 219.1 ± 26.7 |
| hPA-50 | 4970 ± 2880 |

### Example 8

### Neutralizing Activity of Anti-Toxin B Antibody

Each antibody was tested for its neutralizing activity against toxin B in vitro (Babcock et al., Infect. Immun 74: 6339-6347 (2006)). Toxin B was reacted in the presence of varying concentrations of the toxin B-specific monoclonal antibody and then added to cells to evaluate the ability to block the rounding of the toxin B-exposed cells. Human lung fibroblasts IMR-90 were used as the target cells of toxin B. For the activity evaluation, the rate of rounding (%) was determined by the visual observation of the cells and indicated as a cytopathic effect (CPE). The anti-toxin B antibodies used in this activity evaluation were EV029104 and two already publicly available anti-toxin B antibodies MDX1388 (WO2006/121422) and hPA-41 (WO2011/130650). The antibodies MDX1388 and hPA-41 were both prepared on the basis of their reported nucleotide sequences. A human monoclonal antibody (EV2037; WO2010/114105) specific for human cytomegalovirus (HCMV) was used as a negative control. These experiments showed that all of EV029104, MDX1388, and hPA-41 have neutralizing activity (EC50 value) of 100 ng/mL or lower.

### Example 9

### Affinity of Anti-Toxin A Antibody

The affinity of each antibody for toxin A was measured using a Biacore T200® apparatus for detecting the binding interaction between biomolecules by surface plasmon resonance. To a sensor chip CAP, biotinylated toxin A was added, and each antibody was flowed on the chip to measure its binding activity. EV029105a had KD of 1.51×10⁻¹⁰ M. This binding activity was compared with that of the anti-toxin A antibodies 3D8 and hPA-50 measured in the same way as above. The results are shown in Table 3 below. These results demonstrated that among these three anti-toxin A antibodies, EV029105a has the highest affinity.

TABLE 3

| Antibody No. | Kₐ (1/Ms) | K_{d} (1/s) | K_{D} (M) |
|---|---|---|---|
| EV029105a | 6.53 × 10⁵ | 9.89 × 10⁻⁵ | 1.51 × 10⁻¹⁰ |
| 3D8 | 5.87 × 10⁵ | 4.22 × 10⁻⁴ | 7.19 × 10⁻¹⁰ |
| hPA-50 | 8.31 × 10⁵ | 3.49 × 10⁻⁴ | 4.20 × 10⁻¹⁰ |

### Example 10

### Epitope Mapping of Anti-Toxin A Antibody

An epitope on toxin A (SEQ ID NO: 25, GenBank Accession No. P16154) to which each antibody bound was determined by Western blotting. A recombinant E. coli clone expressing four fragments of toxin A, i.e., the enzyme domain (i.e., amino acids 1 to 659 of toxin A), the receptor-binding domain (i.e., amino acids 1853 to 2710 of toxin A), and two intermediate regions (i.e., amino acids 660 to 1256 and 1257 to 1852 of toxin A), was constructed. Each moiety of toxin A (SEQ ID NO: 25) was amplified by PCR from C. difficile 630 strain-derived genomic DNA. These fragments were cloned using pGEX vectors, and BL21 DE3 cells were transformed with the vectors. The expression was induced using IPTG, and Western blotting analysis was conducted with cell lysates of the four fragments of toxin A as antigens. Fragment 1 was the moiety of amino acids 1 to 659; fragment 2 was the moiety of amino acids 660 to 1256; fragment 3 was the moiety of amino acids 1257 to 1852; and fragment 4 was the moiety of amino acids 1853 to 2710. All of EV029105a, 3D8, and hPA-50 reacted with the fragment 4 (receptor-binding domain). In order to find out whether or not EV029105a, 3D8, and hPA-50 bound to the same epitope on the receptor-binding domain, competition assay was conducted using a Biacore T200® apparatus. To a sensor chip CAP, biotinylated toxin A was added, subsequently the EV029105a antibody was added, and finally the 3D8 or hPA-50 antibody was added. The binding of 3D8 and hPA-50 to toxin A was not inhibited, even though EV029105a was first bound with toxin A. These results demonstrated that EV029105a binds to an epitope different from that for 3D8 and hPA-50. These results are shown in FIGS. 3A to 3C.

### Example 11

### Epitope Mapping of Anti-Toxin B Antibody

An epitope on toxin B (SEQ ID NO: 26, GenBank Accession No. Q46034) to which each antibody bound was determined by Western blotting. A recombinant E. coli clone expressing four fragments of toxin B, i.e., the enzyme domain (i.e., amino acids 1 to 546 of toxin B), the receptor-binding domain (i.e., amino acids 1777 to 2366 of toxin B), and two intermediate regions (i.e., amino acids 547 to 1184 and 1185 to 1776 of toxin B), was constructed. Each moiety of toxin B (SEQ ID NO: 26) was amplified by PCR from C. difficile 630 strain-derived genomic DNA. These fragments were cloned using pGEX vectors, and BL21 DE3 cells were transformed with the vectors. The expression was induced using IPTG, and Western blotting analysis was conducted with cell lysates of the four fragments of toxin B as antigens. Fragment 1 was the moiety of amino acids 1 to 546; fragment 2 was the moiety of amino acids 547 to 1184; fragment 3 was the moiety of amino acids 1185 to 1776; and fragment 4 was the moiety of amino acids 1777 to 2366. EV029104 and MDX1388 reacted with the fragment 4 (receptor-binding domain), while hPA-41 reacted with the fragment 1 (enzyme domain). This showed the toxin B-binding site of hPA-41 is different from that of EV029104.

In order to further find out whether or not EV029104 and MDX1388 bound to the same epitope on the receptor-binding domain, competition assay was conducted using a Biacore T200® apparatus. To a sensor chip CAP, biotinylated toxin B was added, subsequently the EV029104 antibody was added, and finally the MDX1388 antibody was added. The binding of MDX1388 to toxin B was not inhibited, even though EV029104 was first bound with the toxin. These results demonstrated that EV029104 binds to an epitope on toxin B different from that for MDX1388. These results are shown in FIG. 4.

### Example 12

### Protective Effect of Anti-Toxin a Antibody Against Mouse Lethality by Toxin A

A test was conducted on whether or not each antibody was able to protect a mouse against the lethal effect of toxin A (WO2006/121422 and WO2011/130650). Each antibody or a control antibody (anti-cytomegalovirus antibody) was intraperitoneally administered (0.165 to 50 μg/head) to 4-week-old Swiss Webster female mice (10 to 15 individuals per group). Approximately 24 hours after the antibody administration, 200 ng of toxin A was intraperitoneally inoculated to each mouse. Approximately 24 hours thereafter, the sign of toxicity was observed and determined on the basis of the survival rate of each animal. The results of these experiments are shown in FIG. 5. For example, EV029105a was confirmed to have a protective effect of 50% even in the administration group that received a low dose of 0.165 μg/head. In the comparison of each anti-toxin A antibody in the 0.5 μg/head administration group, the administration of 3D8 or hPA-50 exhibited a protective effect of 50%, whereas a protective effect as high as 90% was observed in the EV029105a administration group. This in vivo neutralization test showed that EV029105a has high neutralizing activity compared with 3D8 or hPA-50.

### Example 13

### Protective Effects of Anti-Toxin A Antibody and Anti-Toxin B Antibody Against Lethality of Syrian Golden Hamster by C. difficile

Ten Syrian golden hamsters (5-week-old male, approximately 80 g) were used per group. C. difficile (545 strain, ATCC) was orally administered at 100 spores/hamster using a gastric tube. On the day before C. difficile inoculation, 10 mg/kg clindamycin (Nipro Pharma Corp.) was intraperitoneally administered in order to enhance sensitivity to infection. The anti-toxin A antibody EV029105a and the anti-toxin B antibody EV029104 were intraperitoneally administered a total of three times (the day before C. difficile inoculation, the C. difficile inoculation day, and the next day). Results of showing the survival rates of the hamsters in each group are shown in FIGS. 6A to 6B.

FIG. 6A shows the results of comparing antibody administration groups with a group given only clindamycin without the administration of the antibody, wherein the antibody administration groups were set to two groups: a group given EV029105a and EV029104 both at 50 mg/kg per dose; and a group given EV029105a at 10 mg/kg and EV029104 at 50 mg/kg. All of the hamsters in the group without the administration of the antibody died 2 days after the C. difficile inoculation. By contrast, all of the hamsters in the group given EV029105a and EV029104 both at 50 mg/kg survived up to 10 days after the C. difficile inoculation, and all of the hamsters in the group given EV029105a at 10 mg/kg and EV029104 at 50 mg/kg survived up to 5 days after the C. difficile inoculation.

FIG. 6B shows the results of comparing antibody administration groups with a group given only clindamycin without the administration of the antibody, wherein the dose of the antibody was decreased and the antibody administration groups were set to four groups: a group given EV029105a at 10 mg/kg and EV029104 at 10 mg/kg per dose; a group given EV029105a at 10 mg/kg and EV029104 at 2 mg/kg; a group given only EV029105a at 10 mg/kg; and a group given only EV029105a at 2 mg/kg. All of the hamsters in the group without the administration of the antibody died 2 days after the C. difficile inoculation. By contrast, as for the antibody administration groups, the groups that received the combined administration of EV029105a and EV029104 exhibited an EV029104 dose-dependent rise in survival rate and 90% of the hamsters in the group given EV029105a at 10 mg/kg and EV029104 at 10 mg/kg survived even 4 days after the C. difficile inoculation, though all the hamsters in the group given only EV029105a died within 4 days after the C. difficile inoculation. These results demonstrated that the combined administration of EV029105a and EV029104 remarkably protects the hamsters in a dose-dependent manner against the lethal effect of C. difficile.

### INDUSTRIAL APPLICABILITY

The monoclonal antibody of the present invention is useful as a medicine for the treatment of Clostridium difficile infection.

1. An antibody capable of specifically binding to Clostridium difficile toxin A protein and neutralizing its biological activity, or an antigen-binding fragment thereof, comprising:
(i)
(a) heavy chain CDR1 comprising the amino acid sequence of SEQ ID NO: 4 or an amino acid sequence deviated from the amino acid sequence of SEQ ID NO: 4 by deletion, substitution, insertion, and/or addition mutations of one to several amino acid residues,
(b) heavy chain CDR2 comprising the amino acid sequence of SEQ ID NO: 5 or an amino acid sequence deviated from the amino acid sequence of SEQ ID NO: 5 by deletion, substitution, insertion, and/or addition mutations of one to several amino acid residues, and
(c) heavy chain CDR3 comprising the amino acid sequence of SEQ ID NO: 6 or an amino acid sequence deviated from the amino acid sequence of SEQ ID NO: 6 by deletion, substitution, insertion, and/or addition mutations of one to several amino acid residues; and
(ii)
(a) light chain CDR1 comprising the amino acid sequence of SEQ ID NO: 10 or an amino acid sequence deviated from the amino acid sequence of SEQ ID NO: 10 by deletion, substitution, insertion, and/or addition mutations of one to several amino acid residues,
(b) light chain CDR2 comprising the amino acid sequence of SEQ ID NO: 11 or an amino acid sequence deviated from the amino acid sequence of SEQ ID NO: 11 by deletion, substitution, insertion, and/or addition mutations of one to several amino acid residues, and
(c) light chain CDR3 comprising the amino acid sequence of SEQ ID NO: 12 or an amino acid sequence deviated from the amino acid sequence of SEQ ID NO: 12 by deletion, substitution, insertion, and/or addition mutations of one to several amino acid residues.

2. The antibody or an antigen-binding fragment thereof according to claim 1, comprising:
(i)
(a) the amino acid sequence of heavy chain CDR1 comprising the amino acid sequence of SEQ ID NO: 4,
(b) the amino acid sequence of heavy chain CDR2 comprising the amino acid sequence of SEQ ID NO: 5, and
(c) the amino acid sequence of heavy chain CDR3 comprising the amino acid sequence of SEQ ID NO: 6; and
(ii)
(a) the amino acid sequence of light chain CDR1 comprising the amino acid sequence of SEQ ID NO: 10,
(b) the amino acid sequence of light chain CDR2 comprising the amino acid sequence of SEQ ID NO: 11, and
(c) the amino acid sequence of light chain CDR3 comprising the amino acid sequence of SEQ ID NO: 12.

3. The antibody or an antigen-binding fragment thereof according to claim 1, comprising:
(a) a heavy chain variable region (VH) comprising the amino acid sequence of SEQ ID NO: 3 or an amino acid sequence having 95% or higher identity to the amino acid sequence of SEQ ID NO: 3, and
(b) a light chain variable region (VL) comprising the amino acid sequence of SEQ ID NO: 9 or an amino acid sequence having 95% or higher identity to the amino acid sequence of SEQ ID NO: 9.

4. The antibody or an antigen-binding fragment thereof of claim 1, comprising:
(a) a heavy chain variable region (VH) comprising the amino acid sequence of SEQ ID NO: 3, and
(b) a light chain variable region (VL) comprising the amino acid sequence of SEQ ID NO: 9.

5. The antibody or an antigen-binding fragment thereof according to claim 1, wherein the antibody or the antigen-binding fragment thereof is of IgG1 (κ) class (subclass).

6. The antibody or an antigen-binding fragment thereof according to claim 1, wherein the antibody or the antigen-binding fragment thereof has a dissociation constant (KD value) of 1×10⁻⁹ M or lower against the Clostridium difficile toxin A.

7. The antibody or an antigen-binding fragment thereof according to claim 1, wherein the antibody or the antigen-binding fragment thereof has a neutralizing activity (EC50) of 0.05 μg/mL (approximately 0.33 nM) or lower against the Clostridium difficile toxin A in measurement using a human lung fibroblast IMR-90.

8. An antibody capable of specifically binding to Clostridium difficile toxin B protein and neutralizing its biological activity, or an antigen-binding fragment thereof, containing:
(i)
(a) heavy chain CDR1 comprising the amino acid sequence of SEQ ID NO: 16 or an amino acid sequence deviated from the amino acid sequence of SEQ ID NO: 16 by deletion, substitution, insertion, and/or addition mutations of one to several amino acid residues,
(b) heavy chain CDR2 comprising the amino acid sequence of SEQ ID NO: 17 or an amino acid sequence deviated from the amino acid sequence of SEQ ID NO: 17 by deletion, substitution, insertion, and/or addition mutations of one to several amino acid residues, and
(c) heavy chain CDR3 comprising the amino acid sequence of SEQ ID NO: 18 or an amino acid sequence deviated from the amino acid sequence of SEQ ID NO: 18 by deletion, substitution, insertion, and/or addition mutations of one to several amino acid residues; and
(ii)
(a) light chain CDR1 comprising the amino acid sequence of SEQ ID NO: 22 or an amino acid sequence deviated from the amino acid sequence of SEQ ID NO: 22 by deletion, substitution, insertion, and/or addition mutations of one to several amino acid residues,
(b) light chain CDR2 comprising the amino acid sequence of SEQ ID NO: 23 or an amino acid sequence deviated from the amino acid sequence of SEQ ID NO: 23 by deletion, substitution, insertion, and/or addition mutations of one to several amino acid residues, and
(c) light chain CDR3 comprising the amino acid sequence of SEQ ID NO: 24 or an amino acid sequence deviated from the amino acid sequence of SEQ ID NO: 24 by deletion, substitution, insertion, and/or addition mutations of one to several amino acid residues.

9. The antibody or the antigen-binding fragment thereof according to claim 8, comprising:
(i)
(a) the amino acid sequence of heavy chain CDR1 comprising the amino acid sequence of SEQ ID NO: 16,
(b) the amino acid sequence of heavy chain CDR2 comprising the amino acid sequence of SEQ ID NO: 17, and
(c) the amino acid sequence of heavy chain CDR3 comprising the amino acid sequence of SEQ ID NO: 18; and
(ii)
(a) the amino acid sequence of light chain CDR1 comprising the amino acid sequence of SEQ ID NO: 22,
(b) the amino acid sequence of light chain CDR2 comprising the amino acid sequence of SEQ ID NO: 23, and
(c) the amino acid sequence of light chain CDR3 comprising the amino acid sequence of SEQ ID NO: 24.

10. The antibody or the antigen-binding fragment thereof according to claim 8, comprising:
(a) a heavy chain variable region (VH) comprising the amino acid sequence of SEQ ID NO: 15 or an amino acid sequence having 95% or higher identity to the amino acid sequence of SEQ ID NO: 15, and
(b) a light chain variable region (VL) comprising the amino acid sequence of SEQ ID NO: 21 or an amino acid sequence having 95% or higher identity to the amino acid sequence of SEQ ID NO: 21.

11. The antibody or the antigen-binding fragment thereof according to claim 8, comprising:
(a) a heavy chain variable region (VH) comprising the amino acid sequence of SEQ ID NO: 15, and
(b) a light chain variable region (VL) comprising the amino acid sequence of SEQ ID NO: 21.

12. The antibody or the antigen-binding fragment thereof according to claim 8, wherein the antibody or the antigen-binding fragment thereof is of IgG1 (λ) class (subclass).

13. The antibody or the antigen-binding fragment thereof according to claim 8, wherein the antibody or the antigen-binding fragment thereof has a neutralizing activity (EC50) of 0.1 μg/mL (approximately 0.7 nM) or lower against the Clostridium difficile toxin B in measurement using a human lung fibroblast IMR-90.

14. A pharmaceutical composition comprising an antibody or an antigen-binding fragment thereof according to claim 1 and a pharmaceutically acceptable carrier.

15. The pharmaceutical composition according to claim 14, wherein the pharmaceutical composition comprises
(a) a first monoclonal antibody specifically binding to Clostridium difficile toxin A protein or an antigen-binding fragment thereof according to claim 1, and
(b) a second monoclonal antibody specifically binding to Clostridium difficile toxin B protein or an antigen-binding fragment and neutralizing its biological activity, comprising:
(i)
(a) heavy chain CDR1 comprising the amino acid sequence of SEQ ID NO: 16 or an amino acid sequence deviated from the amino acid sequence of SEQ ID NO: 16 by deletion, substitution, insertion, and/or addition mutations of one to several amino acid residues,
(b) heavy chain CDR2 comprising the amino acid sequence of SEQ ID NO: 17 or an amino acid sequence deviated from the amino acid sequence of SEQ ID NO: 17 by deletion, substitution, insertion, and/or addition mutations of one to several amino acid residues, and
(c) heavy chain CDR3 comprising the amino acid sequence of SEQ ID NO: 18 or an amino acid sequence deviated from the amino acid sequence of SEQ ID NO: 18 by deletion, substitution, insertion, and/or addition mutations of one to several amino acid residues; and
(ii)
(a) light chain CDR1 comprising the amino acid sequence of SEQ ID NO: 22 or an amino acid sequence deviated from the amino acid sequence of SEQ ID NO: 22 by deletion, substitution, insertion, and/or addition mutations of one to several amino acid residues,
(b) light chain CDR2 comprising the amino acid sequence of SEQ ID NO: 23 or an amino acid sequence deviated from the amino acid sequence of SEQ ID NO: 23 by deletion, substitution, insertion, and/or addition mutations of one to several amino acid residues, and
(c) light chain CDR3 comprising the amino acid sequence of SEQ ID NO: 24 or an amino acid sequence deviated from the amino acid sequence of SEQ ID NO: 24 by deletion, substitution, insertion, and/or addition mutations of one to several amino acid residues.

16. The pharmaceutical composition according to claim 15, wherein the first and second monoclonal antibodies or an antigen-binding fragments thereof neutralize the Clostridium difficile toxin A and the Clostridium difficile toxin B, respectively, in vitro or in vivo.

17. The pharmaceutical composition according to claim 14, wherein the pharmaceutical composition is intended for the treatment of Clostridium difficile infection.

18. An isolated nucleic acid encoding the amino acid sequence of an antibody or an antigen-binding fragment thereof according to claim 1, an isolated nucleic acid comprising a nucleotide sequence of SEQ ID NO: 1 or 7, or an isolated nucleic acid hybridizing under high stringent conditions to any of these nucleic acids.

19. A vector comprising an isolated nucleic acid according to claim 18 incorporated therein.

20. A host cell comprising a recombinant expression vector according to claim 19 introduced thereinto.

21. An isolated nucleic acid encoding the amino acid sequence of an antibody or an antigen-binding fragment thereof according to claim 8, an isolated nucleic acid comprising a nucleotide sequence of SEQ ID NO: 13 or 19, or an isolated nucleic acid hybridizing under high stringent conditions to any of these nucleic acids.

22. A vector comprising an isolated nucleic acid according to claim 21 incorporated therein.

23. A host cell comprising a recombinant expression vector according to claim 22 introduced thereinto.
